(19) United States
(12) Patent Application Publication
(10) Pub. No.: US 2010/0311683 A1
(43) Pub. Date: Dec. 9, 2010

(54) CYTIDINE ANALOGS FOR TREATMENT OF MYELODYSPLASTIC SYNDROMES

(76) Inventors: C. L. Beach, Lee's Summit, MO (US); Jay Thomas Backstrom, Leawood, KS (US)

Correspondence Address: JONES DAY, 222 E. 41ST. STREET, NEW YORK, NY, 10017, US

(21) Appl. No.: 12/740,636

(22) PCT Filed: Nov. 3, 2008
(86) PCT No.: PCT/US2008/012430

§ 371 Date: Apr. 29, 2010

Related U.S. Application Data
(60) Provisional application No. 60/984,638, filed on Nov. 1, 2007.
(60) Provisional application No. 60/992,781, filed on Dec. 6, 2007.
(60) Provisional application No. 61/034,093, filed on Mar. 5, 2008.
(60) Provisional application No. 61/086,069, filed on Aug. 4, 2008.
(60) Provisional application No. 61/090,852, filed on Aug. 21, 2008.

(51) Int. Cl. A61K 31/7068; A61K 31/706; A61K 31/519; C12Q 1/68; C12Q 1/02; A61P 7/00

(52) U.S. Cl. 514/49; 514/43; 514/267; 435/6; 435/29

(57) ABSTRACT

The present invention provides methods of treating a patient having a higher risk myelodysplastic syndrome, which comprises administering to a patient having a higher risk myelodysplastic syndrome a therapeutically effective amount of a cytidine analog. The cytidine analog includes 5-aza-2′-deoxy cytidine, 5-azacytidine, 5-aza-2′-deoxy- 2′,2′-difluorocytidine, 5-aza-2-40 -deoxy-2′-fluorocytidine, 2′-deoxy-2′,2′-difluorocytidine, cytosine 1-β-D-arabinofuranoside, 2(1H) pyrimidine riboside, 2′-cyclocytidine, arabinofuanosyl-5-azacytidine, dihydro-5-azacytidine, N⁴-octadecyl-cytarabine, and elaidic acid cytarabine

This application claims priority to U.S. Provisional Patent Application No. 60/984,638, filed Nov. 1, 2007; 60/992,781, filed Dec. 6, 2007; 61/034,093, filed Mar. 5, 2008; 61/086,069, filed Aug. 4, 2008; and 61/090,852, filed Aug. 21, 2008; the contents of each of which are incorporated by reference herein in their entireties.

### 1. FIELD

Provided herein are methods for the treatment of myelodysplastic syndromes (“MDS”), e.g., higher risk MDS, using compositions comprising an effective amount of a cytidine analog, including, but not limited to, 5-azacytidine. Also included are methods for improving the overall survival of certain classes of patients having MDS.

### 2. BACKGROUND

Myelodysplastic syndromes (“MDS”) refers to a diverse group of hematopoietic stem cell disorders. MDS is characterized by a cellular marrow with impaired morphology and maturation (dysmyelopoiesis), peripheral blood cytopenias, and a variable risk of progression to acute leukemia, resulting from ineffective blood cell production. See, e.g., The Merck Manual 953 (17th ed. 1999); List et al., 1990, J. Clin. Oncol. 8:1424.

The initial hematopoictic stem cell injury can be from causes such as, but not limited to, cytotoxic chemotherapy, radiation, virus, chemical exposure, and genetic predisposition. A clonal mutation predominates over bone marrow, suppressing healthy stem cells. In the early stages of MDS, the main cause of cytopenias is increased programmed cell death (apoptosis). As the disease progresses and converts into leukemia, gene mutation rarely occurs and a proliferation of leukemic cells overwhelms the healthy marrow. The disease course differs, with some cases behaving as an indolent disease and others behaving aggressively with a very short clinical course that converts into an acute form of leukemia.

An international group of hematologists, the French-American-British (FAB) Cooperative Group, classified MDS into five subgroups, differentiating them from acute myeloid leukemia. See, e.g., The Merck Manual 954 (17th ed. 1999); Bennett J. M., et al., Ann. Intern. Med. 1985 October, 103(4): 620-5; and Besa E. C., Med. Clin. North Am. 1992 May, 76(3): 599 617. An underlying trilineage dysplastic change in the bone marrow cells of the patients is found in all subtypes. Information is available regarding the pathobiology of MDS, certain MDS classification systems, and particular methods of treating and managing MDS. See, e.g., U.S. Pat. No. 7,189,740 (issued Mar. 13, 2007), which is incorporated by reference herein in its entirety.

Nucleoside analogs have been used clinically for the treatment of viral infections and proliferative disorders for decades. Most of the nucleoside analog drugs are classified as antimetabolites. After they enter cells, nucleoside analogs are successively phosphorylated to nucleoside 5′-monophosphates, 5′-diphosphates, and 5′-triphosphates. In most cases, nucleoside triphosphates are the chemical entities that inhibit DNA or RNA synthesis, either through a competitive inhibition of polymerases or through incorporation of modified nucleotides into DNA or RNA sequences. Nucleosides may act also as their diphosphates.

5-Azacytidine (also known as azacitidine and 4-amino-1-β-D-ribofuranosyl-1,3,5-triazin-2(1H)-one; Nation Service Center designation NSC-102816; CAS Registry Number 320-67-2) has undergone NCI-sponsored trials for the treatment of MDS. See, e.g., Kornblith et al., J. Clin. Oncol. 20(10): 2441-2452 (2002); Silverman et al., J Clin. Oncol. 20(10): 2429-2440 (2002). 5-Azacytidine may be defined as having a molecular formula of C₈H₁₂N₄O₅, a relative molecular weight of 244.21 and a structure of:

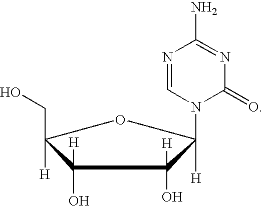

Azacitidine (also referred to as 5-azacytidine herein) is a nucleoside analog, more specifically a cytidine analog. 5-Azacytidine is an antagonist of its related natural nucleoside, cytidine. 5-Azacytidine, as well as decitabine, i.e., 5-aza-2′-deoxycytidine, are antagonists of decitabine's related natural nucleoside, deoxycytidine. The only structural difference between the analogs and their related natural nucleosides is the presence of nitrogen at position 5 of the cytosine ring in place of oxygen.

Other members of the class of deoxycytidine and cytidine analogs include arabinosylcytosine (Cytarabine), 2′-deoxy-2′,2′-difluorocytidine (Gemcitabine), 5-aza-2′-deoxycytidine (Decitabine), 2(1H)-pyrimidine-riboside (Zebularine), 2′,3′-dideoxy-5-fluoro-3′-thiacytidine (Emtriva), N⁴-pentyloxycarbonyl-5′-deoxy-5-fluorocytidine (Capecitabine), 2′-cyclocytidine, arabinofuanosyl-5-azacytidine, dihydro-5-azacytidine, N⁴-octadecyl-cytarabine, elaidic acid cytarabine, and cytosine 1-β-D-arabinofuranoside (ara-C).

A need remains for more effective methods and compositions which provide, e.g., increased survival to higher risk MDS patients.

### 3. SUMMARY

Embodiments herein provide methods for the treatment of myelodysplastic syndromes (MDS) using compositions comprising an effective amount of a cytidine analog, including, but not limited to, 5-azacytidine. Particular embodiments provide methods for treating patients with higher risk MDS using 5-azacytidine. Particular embodiments provide methods for improving the overall survival of patients having MDS, e.g., higher risk MDS. Particular embodiments provide alternative dosing regimens for treating MDS. Particular embodiments provide methods for treating certain subgroups of patients with higher risk MDS, e.g., patients with −7/del(7q). Particular embodiments provide methods for treating elderly patients with acute myelogenous leukemia (“AML”). Particular embodiments provide methods for ameliorating certain adverse events (“AEs”) in patients with MDS, e.g., higher risk MDS. Particular embodiments provide methods for treating patients having MDS, e.g., higher risk MDS, using specific numbers of azacytidine treatment cycles. Particular embodiments provide methods of treating patients who meet the WHO criteria for AML using azacytidine. Particular embodiments provide methods of using IWG responses of complete remission, partial remission, hematologic improvement, and/or stable disease as predictors of overall response in patients with MDS, e.g., higher risk MDS. Particular embodiments provide using azacytidine as maintenance therapy. Particular embodiments provide using DNA and/or RNA methylation as biomarkers for overall survival in patients with MDS, e.g., higher risk MDS.

### 4. BRIEF DESCRIPTION OF THE DRAWINGS

FIG. 1 represents a graph showing overall survival in the intent to treat population (ITT, higher risk MDS patients) of 5-azacytidine compared to conventional care regimens (CCR).

FIG. 2 represents a study design for the Phase III azacitidine survival study.

FIG. 3 represents a graph showing overall survival in the intent to treat population (higher risk MDS patients) of 5-azacytidine compared to conventional care regimens.

FIG. 4 represents the Hazard Ratio and 95% CI for overall survival in predefined subgroups.

FIG. 5 represents time to transform to AML-ITT Population, showing numbers at risk over time.

FIG. 6 represents time to transform to AML-ITT Population comparing the azacitidine group with the CCR group, showing difference of 13.7 months in time to transformation

FIG. 7 represents a study design for a multi-center, randomized, open-label, Phase II MDS study.

FIG. 8 represents a chart showing the grouping of patients in the ITT cohort for the Phase III azacitidine survival study.

FIG. 9 represents the ITT cohort for the multi-center, randomized, open-label, Phase II study.

FIG. 10 represents RBC transfusion independence in baseline-dependent patients in the Phase II study.

FIG. 11 represents investigator's pre-selection, randomization, and disposition of patients for the Phase III azacitidine survival study.

FIG. 12 represents hazard ratio and 95% CI for overall survival: azacitidine vs. CCR (ITT population).

FIG. 13 represents overall survival of the azacitidine subgroup and the LDAC subgroup.

FIG. 14 represents effect of AZA vs. CCR on overall survival in patients over 75 years of age.

FIG. 15 represents overall survival of the Aza subgroup vs. the CCR subgroup in WHO AML patients.

FIG. 16 represents methylation results.

### 5. DETAILED DESCRIPTION

Embodiments provided herein are methods of treatments with a pharmaceutical composition comprising a cytidine analog, particularly, 5-azacytidine, providing particular benefit to the population of patients stratified into the higher risk groups of myelodysplastic syndromes (MDS) by conventional scoring systems, as measured by improved survival of this population upon treatment with a cytidine analog, e.g., azacitidine.

Accordingly, in one embodiment, provided herein is a method of treating a patient diagnosed with a higher risk MDS, the method comprising treating the patient diagnosed with a higher risk MDS with an effective amount of a composition comprising a cytidine analog.

In one embodiment, the cytidine analog includes any moiety which is structurally related to cytidine or deoxycytidine and functionally mimics and/or antagonizes the action of cytidine or deoxycytidine. These analogs may also be called cytidine derivatives herein. In one embodiment, cytidine analog includes 5-aza-2′-deoxycytidine(decitabine), 5-azacytidine, 5-aza-2′-deoxy-2′,2′-difluorocytidine, 5-aza-2′-deoxy-2′-fluorocytidine, 2′-deoxy-2′,2′-difluorocytidine (also called gemcitabine), or cytosine 1-β-D-arabinofuranoside (also called ara-C), 2(1H)-pyrimidine-riboside (also called zebularine), 2′-cyclocytidine, arabinofuanosyl-5-azacytidine, dihydro-5-azacytidine, N⁴-octadecyl-cytarabine, and elaidic acid cytarabine. In one embodiment, cytidine analog includes 5-azacytidine and 5-aza-2′-deoxycytidine. The definition of cytidine analog used herein also includes mixtures of cytidine analogs.

Cytidine analogs may be synthesized by methods known in the art. In one embodiment, methods of synthesis include methods as disclosed in U.S. Ser. No. 10/390,526 (U.S. Pat. No. 7,038,038); U.S. Ser. No. 10/390,578 (U.S. Pat. No. 6,887,855); U.S. Ser. No. 11/052615 (U.S. Pat. No. 7,078,518); U.S. Ser. No. 10/390,530 (U.S. Pat. No. 6,943,249); and U.S. Ser. No. 10/823,394, all incorporated by reference herein in their entireties.

In one embodiment, an effective amount of a cytidine analog to be used is a therapeutically effective amount. In one embodiment, the amounts of a cytidine analog to be used in the methods provided herein and in the oral formulations include a therapeutically effective amount, typically, an amount sufficient to cause improvement in at least a subset of patients with respect to symptoms, overall course of disease, or other parameters known in the art. Therapeutic indications are discussed more fully herein below. Precise amounts for therapeutically effective amounts of the cytidine analog in the pharmaceutical compositions will vary depending on the age, weight, disease, and condition of the patient. For example, pharmaceutical compositions may contain sufficient quantities of a cytidine analog to provide a daily dosage of about 10 to 150 mg/m² (based on patient body surface area) or about 0.1 to 4 mg/kg (based on patient body weight) as single or divided (2-3) daily doses. In one embodiment, dosage is provided via a seven day administration of 75 mg/m² subcutaneously, once every twenty-eight days, for as long as clinically necessary. In one embodiment, up to 9 or more 28-day cycles are administered. Other methods for providing an effective amount of a cytidine analog are disclosed in, for example, “Colon-Targeted Oral Formulations of Cytidine Analogs”, U.S. Ser. No. 11/849,958, which is incorporated by reference herein in its entirety.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

Acute myeloid leukemia (AML) is the most common type of acute leukemia that occurs in adults. Several inherited genetic disorders and immunodeficiency states are associated with an increased risk of AML. These include disorders with defects in DNA stability, leading to random chormosomal breakage, such as Bloom's syndrome, Fanconi's anemia, Li-Fraumeni kindreds, ataxia-telangiectasia, and X-linked agammaglobulinemia.

Acute promyelocytic leukemia (APML) represents a distinct subgroup of AML. This subtype is characterized by promyelocytic blasts containing the 15;17 chromosomal translocation. This translocation leads to the generation of the fusion transcript comprised of the retinoic acid receptor and a sequence PML.

Acute lymphoblastic leukemia (ALL) is a heterogenerous disease with distinct clinical features displayed by various subtypes. Reoccurring cytogenetic abnormalities have been demonstrated in ALL. The most common cytogenetic abnormality is the 9;22 translocation. The resultant Philadelphia chromosome represents poor prognosis of the patient.

Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

The myelodysplastic syndromes (MDS) are heterogeneous clonal hematopoietic stem cell disorders grouped together, because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia. MDS affects approximately 40,000-50,000 people in the U.S. and 75,000-85,000 patients in Europe. The majority of people with higher risk MDS eventually experience bone marrow failure. Up to 50% of MDS patients succumb to complications, such as infection or bleeding, before progressing to acute myeloid leukemia (AML). MDS patients have a median survival of four months to five years depending on risk stratification. Higher risk patients have a median survival of five to 14 months. Altering the natural history of the disease and providing increased survival is one of the most important treatment goals in higher risk MDS.

In one embodiment, MDS is a condition to be treated with methods provided herein, and includes the following MDS subtypes: refractory anemia, refractory anemia with ringed sideroblasts (if accompanied by neutropenia or thrombocytopenia or requiring transfusions), refractory anemia with excess blasts, refractory anemia with excess blasts in transformation, and chronic myelomonocytic leukemia. In another embodiment, the condition to be treated is higher risk MDS.

In classifying a patient's disease as “higher risk MDS” (also referred to herein as, e.g., “higher-risk MDS,” “high risk MDS” and “high-risk MDS”), methods known in the art can be used by the skilled person in order to classify a patient's disease as “higher risk” MDS. Such methods include, e.g., the FAB system, the WHO system, and IPSS, as discussed herein below (See, e.g., Bennett J. M., A comparative review of classification systems in myelodysplastic syndromes (MDS), Semin. Oncol. 2005 August; 32(4 Suppl 5):S3-10; Bennett et al., Br. J. Haematol. 1982, 51:189-99; Harris et al., J. Clin. Oncol. 1999, 17(12):3835-49; Greenberg et al., Blood 1997, 89(6), 2079-98). Other methods for such assessment may lie within the knowledge or expertise of the skilled person, and methods provided herein include such a skilled person's assessment.

The skilled person knows that experience has shown that certain disease factors affect a person's prognosis—his or her chances of long-term survival and risk of developing AML. Researchers use these factors to classify MDS into types. In one embodiment, the system to classify MDS is the FAB system, so-called because it was developed by a team of French, American and British researchers. In the FAB system, there are five types of MDS. The FAB system uses several disease factors to classify MDS. One important factor is the percent of blasts in the bone marrow (Table 1). A higher percent of blasts is linked to a higher likelihood of developing AML and a poorer prognosis. The two more common types of MDS are refractory anemia (RA) and refractory anemia with ringed sideroblasts (RARS). These are also the less severe forms of MDS. They have a lower risk of turning into AML. Some patients with these forms of MDS may live with few symptoms and need little treatment for many years.

The other types of MDS tend to be more severe and more difficult to treat successfully. The refractory anemia with excess blasts (RAEB) and refractory anemia with excess blasts in transformation (RAEB-t) forms of MDS also have a high risk of turning into AML.

TABLE 1
MDS Types in the FAB System

| | Percent of blasts in marrow |
|---|---|
| Type of MDS | (less than 5% is normal) |
| Refractory anemia (RA) | Less than 5% (normal amount) |
| Refractory anemia with ringed | Less than 5% (normal amount), |
| sideroblasts (RARS) | plus more than 15% of |
| | abnormal red blood cells |
| | called ringed sideroblasts |
| Refractory anemia with excess blasts | 5% to 20% |
| (RAEB) | |
| Refractory anemia with excess blasts in | 21% to 30% |
| transformation (RAEB-T) | |
| Chronic myelomonocytic leukemia | 5% to 20%, plus a large number |
| (CMML) | of a type of white blood cell |
| | called monocytes |

In another embodiment, a system for defining types of MDS is the newer World Health Organization (WHO) system which divides MDS into eight types. (See, e.g., Muller-Berndorff, et al., Ann. Hematol. 2006 August; 85(8):502-13.) In certain embodiments, a skilled person may use either the FAB or WHO system to determine the type of MDS.

In another embodiment, individual prognosis is determined using the international prognostic scoring system (IPSS). The IPSS risk score describes the risk that a person's disease will develop into AML or become life-threatening. A doctor may use the IPSS risk score along with the MDS type to plan treatment. The IPSS risk score is based on three factors that have been shown to affect a patient's prognosis:

(1) The percent of cells in the bone marrow that are blasts.

(2) Whether one, two or all three types of blood cells are low (also called cytopenias). The three types are red blood cells, white blood cells, and platelets.

(3) Changes in the chromosomes of bone marrow blood cells. This may be called cytogenetics (the study of chromosome abnormalities). It may also be called the karyotype (a picture of the chromosomes that shows whether they are abnormal).

A person may have an IPSS risk score of low, intermediate-1, intermediate-2 or high risk. Doctors can use the risk score to plan treatment. Someone with low-risk disease may be likely to survive for years with few symptoms. That person may need less intense treatment. Someone with intermediate-1, intermediate-2 or high-risk disease may be likely to survive only if he or she receives aggressive treatment, such as a transplant.

In one embodiment, a higher risk patient is treated by the methods provided herein. In one embodiment, a patient defined as a higher risk MDS patient includes those whose disease is assessed as any one or more of the following: RAEB, RAEB-T, or CMML (10-29% marrow blasts) under FAB or with an IPSS of Intermediate-2 or High.

In one embodiment, dosing schedules for the compositions and methods provided herein, for example, can be adjusted to account for the patient's characteristics and disease status. Appropriate dose will depend on the disease state being treated. In some cases, dosing schedules include daily doses, and in others, selected days of a week, month or other time interval. In one embodiment, the drug will not be given more than once per day. In one embodiment, dosing schedules for administration of pharmaceutical compositions include the daily administration to a patient in need thereof Dosing schedules may mimic those that are used for non-oral formulations of a cytidine analog, adjusted to maintain, for example, substantially equivalent therapeutic concentration in the patient's body.

In certain embodiments, appropriate biomarkers may be used to evaluate the drug's effects on the disease state and provide guidance to the dosing schedule. For example, particular embodiments herein provide a method of determining whether a patient diagnosed with MDS has an increased probability of obtaining a greater benefit from treatment with a cytidine analog by assessing the patient's nucleic acid methylation status. In particular embodiments, the cytidine analog is azacitidine. In particular embodiments, the nucleic acid is DNA or RNA. In particular embodiments, the greater benefit is an overall survival benefit. In particular embodiments, the methylation status is examined in one or more genes, e.g., genes associated with MDS or AML. Specific embodiments involve methods for determining whether baseline DNA methylation levels influence overall survival in patients with MDS (e.g., higher risk MDS) treated with azacitidine. Specific embodiments provide methods for determining whether gene promoter methylation levels influence overall survival in patients with MDS (e.g., higher risk MDS).

For example, specific embodiments herein provide methods for evaluating the influence of gene methylation on prolonged survival in patients with MDS (e.g., higher risk MDS). In particular embodiments, such evaluation is used to predict overall survival in patients with MDS (e.g., higher risk MDS), e.g., upon treatment with azacitidine. In particular embodiments, such evaluation is used for therapeutic decision-making. In specific embodiments, such therapeutic decision-making includes planning or adjusting a patient's treatment, e.g., the dosing regimen, amount, and/or duration of azacitidine administration.

Certain embodiments provide methods of identifying individual patients diagnosed with MDS having an increased probability of obtaining an overall survival benefit from azacitidine treatment, using analysis of methylation levels, e.g., in particular genes. In specific embodiments, lower levels of nucleic acid methylation are associated with an increased probability of obtaining improved overall survival following azacitidine treatment. In particular embodiments, the increased probability of obtaining improved overall survival following azacitidine treatment is at least a 5% greater probability, at least a 10% greater probability, at least a 20% greater probability, at least a 30% greater probability, at least a 40% greater probability, at least a 50% greater probability, at least a 60% greater probability, at least a 70% greater probability, at least an 80% greater probability, at least a 90% greater probability, at least at least a 100% greater probability, at least a 125% greater probability, at least a 150% greater probability, at least a 175% greater probability, at least a 200% greater probability, at least a 250% greater probability, at least a 300% greater probability, at least a 400% greater probability, or at least a 500% greater probability of obtaining improved overall survival following azacitidine treatment. In particular embodiments, the greater probability of obtaining improved overall survival following azacitidine treatment is a greater probability as compared to the average probability of a particular comparison population of patients diagnosed with MDS. In specific embodiments, the comparison population is a group of patients classified with a particular myelodysplastic subtype, as described herein. In one embodiment, the comparison population consists of patients having higher risk MDS. In particular embodiments, the comparison population consists of a particular IPSS cytogenetic subgroup.

In particular embodiments, nucleic acid (e.g., DNA or RNA) hypermethylation status may be determined by any method known in the art. In certain embodiments, DNA hypermethylation status may be determined using the bone marrow aspirates of patients diagnosed with MDS, e.g., by using quantitative real-time methylation specific PCR (“qMSP”). In certain embodiments, the methylation analysis may involve bisulfite conversion of genomic DNA. For example, in certain embodiments, bisulfite treatment of DNA is used to convert non-methylated CpG sites to UpG, leaving methylated CpG sites intact. See, e.g., Frommer, M., et al., Proc. Nat'l Acad. Sci. USA 1992, 89:1827-31. Commercially available kits may be used for such bisulfite treatment. In certain embodiments, to facilitate methylation PCR, primers are designed as known in the art, e.g., outer primers which amplify DNA regardless of methylation status, and nested primers which bind to methylated or non-methylated sequences within the region amplified by the first PCR. See, e.g., Li et al., Bioinformatics 2002, 18:1427-31. In certain embodiments, probes are designed, e.g., probes which bind to the bisulfite-treated DNA regardless of methylation status. In certain embodiments, CpG methylation is detected, e.g., following PCR amplification of bisulfite-treated DNA using outer primers. In certain embodiments, amplified product from the initial PCR reaction serves as a template for the nested PCR reaction using methylation-specific primers or non-methylation-specific primers. In certain embodiments, a standard curve is established to determine the percentage of methylated molecules in a particular sample. Methods for detecting nucleic acid methylation (e.g., RNA or DNA methylation) are known in art. See, e.g., Laird, P. W., Nature Rev. Cancer 2003, 3:253-66; Belinsky, S. A., Nature Rev. Cancer 2004, 4:1-11.

In certain embodiments, statistical analyses are performed to assess the influence of particular methylation levels with the potential benefit of treatment with a particular cytidine analog. In certain embodiments, the influence of methylation on overall survival is assessed, e.g., using Cox proportional hazards models and Kaplan-Meier (KM) methodology.

In certain embodiments, any gene associated with MDS and/or AML may be examined for its methylation status in a patient. Particular genes include, but are not limited to, CKDN2B (p15), SOCS1, CDH1 (E-cadherin), TP73, and CTNNA1 (alpha-catenin). Particular genes associated with MDS and/or AML, which would be suitable for use in the methods disclosed here, are known in the art.

In another embodiment, provided herein is a method of selecting a patient diagnosed with MDS for treatment with 5-azacytidine, comprising assessing a patient diagnosed with MDS for having higher risk, and selecting a patient for treatment with 5-azacytidine where the patient's MDS is assessed as having higher risk. In another embodiment, provided herein is a method to improve survival in a patient population with higher risk MDS, the method comprising treating at least one patient diagnosed with a higher risk MDS with an effective amount of a composition comprising a cytidine analog.

Certain embodiments herein provide methods for the treatment of MDS. In certain embodiments, the methods comprise providing for the survival of an MDS patient beyond a specific period of time by administering a specific dose of azacitidine for at least a specific number of cycles of azacitidine treatment. In particular embodiments, the contemplated specific period of time for survival is, e.g., beyond 10 months, beyond 11 months, beyond 12 months, beyond 13 months, beyond 14 months, beyond 15 months, beyond 16 months, beyond 17 months, beyond 18 months, beyond 19 months, or beyond 20 months. In particular embodiments, the contemplated specific number of cycles administered is, e.g., at least 7, at least 8, at least 9, at least 10, at least 11, at least 12, at least 13, at least 14, or at least 15 cycles of azacitidine treatment. In particular embodiments, the contemplated treatment is administered, e.g., 1, 2, 3, 4, 5, 6, 7, 8, 9, 10, 11, 12, 13, or 14 days out of a 28-day period. In particular embodiments, the contemplated specific azacitidine dose is, e.g., at least at least 10 mg/day, at least 20 mg/day, at least 30 mg/day, at least 40 mg/day, at least 50 mg/day, at least 55 mg/day, at least 60 mg/day, at least 65 mg/day, at least 70 mg/day, at least 75 mg/day, at least 80 mg/day, at least 85 mg/day, at least 90 mg/day, at least 95 mg/day, or at least 100 mg/day. In particular embodiments, the dosing is performed, e.g., subcutaneously or intravenously. One particular embodiment herein provides a method for obtaining the survival of an MDS patient beyond 15 months by administering at least 9 cycles of azacitidine treatment. One particular embodiment herein provides administering the treatment for 7 days out of each 28-day period. One particular embodiment herein provides a dosing regimen of 75 mg/m² subcutaneously or intravenously, daily for 7 days.

### 6. EXAMPLES

The following examples are provided by way of illustration, not limitation.

### 6.1 Example 1

This phase III randomized trial assessed the effect of azacitidine on prolonging overall survival in patients with higher risk MDS compared with 3 other frequently used conventional care regimens.

A phase III, international, multi-center, prospective, randomized, controlled, parallel group trial was conducted and demonstrated prolonged overall survival in higher risk MDS patients as compared to conventional care regimens and best supportive care. (This study is referred to herein as the “AZA-001” study). The primary study objective and endpoint were overall survival (OS), comparing azacitidine and conventional care regimens. Secondary objectives and endpoints included time to transformation to acute myeloid leukemia (AML), red blood cell transfusion independence, hematologic responses and improvement, infections requiring IV therapy, and safety.

Eligible patients were 18 years or older with higher risk MDS, defined as an IPSS of Intermediate-2 or High and FAB-defined RAEB, RAEB-T, or non-myeloproliferative chronic myelomonocytic leukemia (CMML), using modified FAB criteria (blood monocytes greater than 1×10⁹/L, dysplasia in 1 or more myeloid cell lines, 10%-29% marrow blasts, and a white blood count below 13×10⁹/L). Patients were to have an Eastern Cooperative Oncology Group (ECOG) performance status of 0-2 and life expectancy of 3 months or more. Patients with secondary therapy-related MDS, prior azacitidine treatment, or eligibility for allogenetic stem cell transplantation were excluded.

The Phase III, international, multi-center, randomized, controlled, parallel-group trial was conducted in accordance with the Declaration of Helsinki. All patients provided written informed consent, and the study was approved by the institutional review boards at all participating study sites. Enrollment to the trial and monitoring was conducted by site investigators and central pathology reviewers with standardized central review of cytogenetic data. An independent Data Safety Monitoring Board reviewed safety data and conducted blinded review of a scheduled interim analysis.

Patients were randomized to 1 of 2 treatment groups: azacitidine plus best supportive care (BSC) or conventional care regimens (CCR) plus BSC. Patients were randomized 1:1 to receive azacitidine or CCR. Prior to randomization, investigators pre-selected (based on age, health and disease status, co-morbidities, etc.) the most appropriate one of three conventional CCR groups for higher risk MDS patients, which the patients then received if randomized to CCR. Patients randomized to azacitidine received azacitidine regardless of CCR selection. This pre-randomization step was performed to enable meaningful comparisons of CCR subgroups with relevant azacitidine-treated subgroups. No crossover was allowed in this trial and administration of erythropoietin or darbepoetin was prohibited. Balanced enrollment across treatments was ensured using blocked randomization with patients stratified by FAB subtype and IPSS risk group.

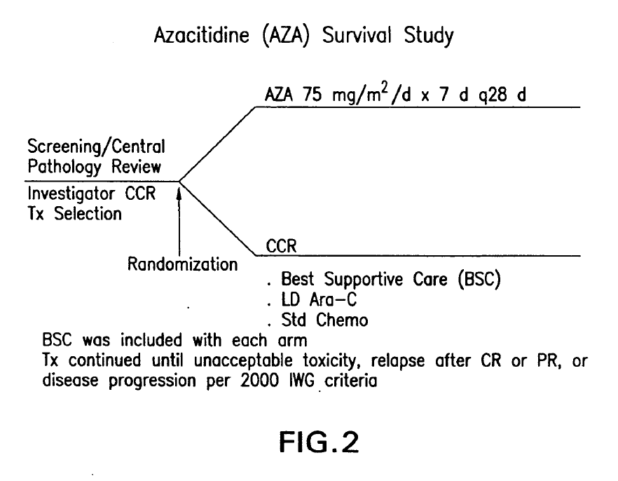

During the treatment phase of the trial, all regimens were continued until study end or patient discontinuation due to relapse, disease progression, unacceptable toxicity, or transformation to AML (defined as 30% or greater bone marrow blasts). Azacitidine was administered subcutaneously at 75 mg/m²/day for 7 days every 28 days (delayed as needed until cell line recovery), which constituted one cycle of therapy, for at least 6 cycles until study end unless treatment was discontinued due to unacceptable toxicity, relapse after response, or disease progression. The CCR group consisted of 3 treatment regimens administered until study end or treatment discontinuation: BSC only (including blood product transfusions, antibiotics, with G-CSF for neutropenic infection); low-dose ara-C (LDara-C): 20 mg/m²/day subcutaneously for 14 days, every 28-42 days (delayed as needed until cell line recovery) for at least 4 cycles; or intensive chemotherapy, i.e. induction with ara-C 100-200 mg/m²/day by continuous intravenous infusion for 7 days plus 3 days of intravenous daunorubicin (45-60 mg/m²/day), idarubicin (9-12 mg/m²/day), or mitoxantrone (8-12 mg/m²/day). Patients with complete or partial remission after induction (defined by IWG criteria for AML, see e.g., J. Clin. Oncol. 2003, 21(24):4642-9) received 1-2 consolidation courses with reduced doses of the cytotoxic agents used for induction, followed by BSC only. All patients could receive BSC as needed. After treatment discontinuation, all patients were followed until death or end of study (12 months following randomization of the last patient). FIG. 2 shows the study design.

All efficacy analyses used the intent-to-treat (ITT) population. Safety analyses were performed on the safety population (all patients who received at least 1 dose of study drug and 1 or more post-dose safety assessments). The primary trial endpoint was overall survival (time from randomization until death from any cause), analyzed for the ITT group comparing the azacitidine group and the CCR group, and for predefined subgroups based on age, gender, FAB, IPSS (Int-2, high), IPSS cytogenetics (good, intermediate, and poor) and −7/del(7q) cytogenetic abnormality, IPSS cytopenias (0/1 and 2/3), WHO classification, karyotype, and lactic dehydrogenase (LDH). The primary assessment of overall survival used the ITT population and compared azacitidine with the combined CCR group. A secondary analysis compared overall survival of azacitidine subgroups (the 3 CCR subgroups of patients who were randomized to azacitidine) with the corresponding CCR subgroups (patients in the corresponding CCR subgroups, who were randomized to CCR).

Secondary efficacy endpoints were transformation to AML (from randomization until AML transformation [30% bone marrow blast count or greater]), hematologic response and improvement assessed using IWG 2000 criteria for MDS (See e.g., Cheson et al., Blood 2000, 96(12):3671-4), red blood cell (RBC) transfusion independence (absence of transfusions during 56 consecutive days), infections requiring intravenous antimicrobials (analyzed from randomization to 28 days post last study visit), and adverse events. Bone marrow samples were collected every 16 weeks during active treatment and as clinically indicated during follow-up. Infections requiring intravenous antimicrobials were counted from randomization to last study visit. Adverse events were assessed using the National Cancer Institute's Common Toxicity Criteria, Version 2.0.

Time to event was studied using the Kaplan-Meier method; treatment comparisons were made using stratified log-rank tests and Cox proportional-hazards models. All statistical tests were two-sided without correction for multiple testing.

Efficacy analyses included all patients randomized according to the ITT principle. Overall survival was defined as the time from randomization until death from any cause. Patients for whom death was not observed were censored at the time of last follow-up. Time to transformation to AML was measured from randomization to development of 30% or greater bone marrow blasts. Patients for whom AML transformation was not observed were censored at the time of last adequate bone marrow sample. Randomization and analyses were stratified on FAB subtype and IPSS risk group. Time-to-event curves were estimated according to the Kaplan-Meier method (See e.g., Kaplan et al., J. Am. Stat. Assoc. 1958, 53; 457-81) and compared using stratified log-rank tests (primary analysis). Stratified Cox proportional hazards regression models (See e.g., Cox, J. Royal Stat. Soc. B, 1972, 34; 184-92) were used to estimate hazard ratios and associated 95% confidence intervals (CI). The primary analysis of overall survival between the azacitidine and combined CCR groups used the stratified Cox proportional hazards model without any covariate adjustments to estimate the hazard ratio. Cox proportional hazards regression with stepwise selection was used to assess the baseline variables of sex, age, time since original MDS diagnosis, ECOG performance status, number of RBC transfusions, number of platelet transfusions, hemoglobin, platelets, absolute neutrophil count, LDH, bone marrow blast percentage, and presence or absence of cytogenetic −7/del(7q) abnormality. The final model included ECOG performance status, LDH, hemoglobin, number of RBC transfusions and presence or absence of cytogenetic −7/del(7q) abnormality. Secondary analyses used the final Cox proportional hazards model. The consistency of treatment effect across subgroups was assessed by the difference in likelihood ratio between the full model with treatment, subgroup and treatment-by-subgroup interaction, and the reduced model without the interaction.

Response rates (overall response, transfusion independence, and hematological improvement) were compared between the azacitidine and CCR groups using Fisher's exact test. The rate of infection requiring intravenous antimicrobials was computed as the number of observed infections requiring intravenous antimicrobials divided by the total number of patient-years of follow-up. The relative risk was computed by dividing the azacitidine rate by the CCR rate. The relative risks across the 4 strata were tested for homogeneity using the Breslow-Day test (See e.g., Breslow et al., Chapter 3: Comparisons Among Exposure Groups. In: Heseltine E. ed. Statistical Methods in Cancer Research Volume II-The Design and Analysis of Cohort Studies. Lyon: IARC Scientific Publications; 1987:82-119). The Mantel-Haenszel estimate of the common relative risk, the associated 95% CI, and the test that it equals unity were computed (See e.g., Mantel, Cancer Chemotherapy Reports, 1966, 50(3):163-70). This study was designed with 90% power—based on a log rank analysis—to detect a hazard ratio of 0.60 for overall survival in the azacitidine group compared with the CCR group with a two-sided alpha of 0.05. The protocol specified that approximately 354 patients were to be randomized over 18 months and then monitored for at least 12 months of treatment and follow-up, resulting in at least 167 deaths over the 30 month trial period. Recruitment, however, necessitated a longer study period that lasted 42 months with 195 deaths that resulted in a 95% power under the design assumptions of the study. The interim analysis was conducted using an O'Brien-Fleming monitoring boundary and Lan-DeMets alpha spending function to control the overall alpha at 0.05 (See e.g., Lan et al., Biometrika 1983, 70(3):659-63).

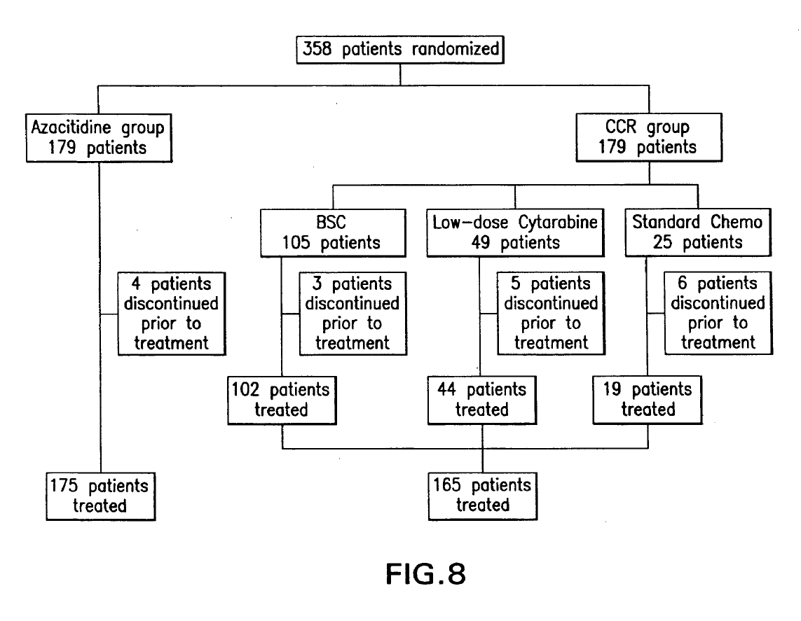

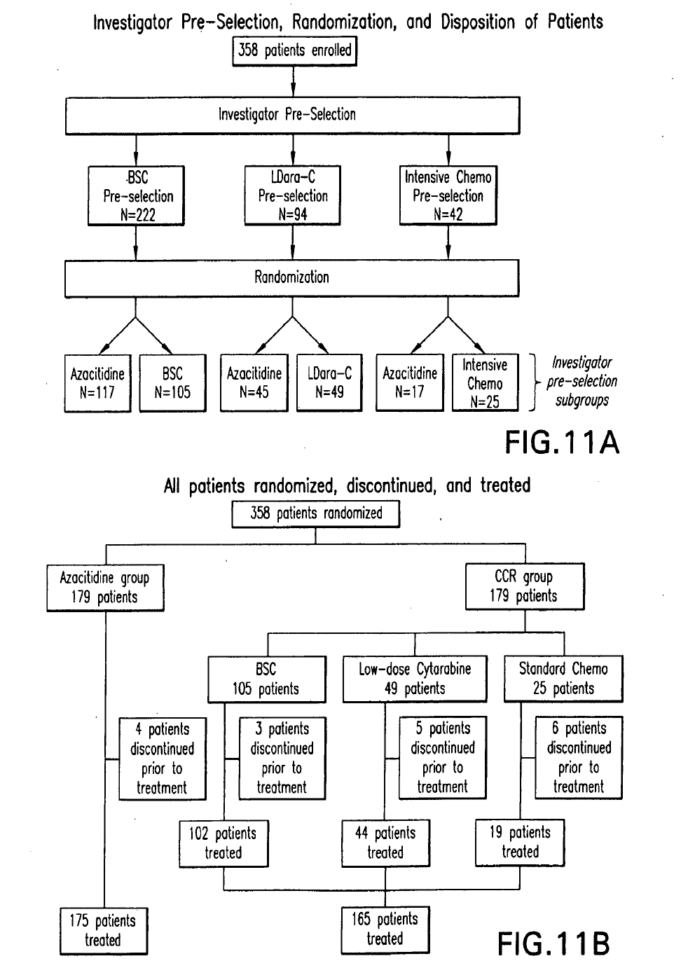

358 Patients (ITT population, 98% Caucasian, 70% male) at 79 sites were randomized: 179 to azacitidine and 179 to CCR (105 to BSC 59%, 49 to LDara-C 27%, and 25 to intensive chemotherapy 14%, FIGS. 8 and 11). Median age was 69 years (range: 38-88) with 258 (72%) patients aged 65 years or older. Baseline demographic and disease characteristics were well balanced between the azacitidine and CCR combined and between azacitidine and the 3 CCR regimens (Table 2A and 2B). As expected, patients in the intensive chemotherapy group were younger. At baseline, 95% of patients were higher risk: RAEB (58%), RAEB-T (34%), CMML (3%), and other (5%). By IPSS, 87% were higher risk: Int-2 (41%), High (47%), and 13% indeterminate/other. Additionally, 32% of patients were classified as WHO AML (marrow blast count, 20%-30%). Upon IRC review, 10 and 5 patients, respectively, in the azacitidine and CCR groups had received prior radiation, chemotherapy, or cytotoxic therapies for non-MDS conditions, which constituted protocol deviations. Azacitidine was administered for a median of 9 cycles (range 1 to 39) with 86% of patients remaining on the 75 mg/m²/day dose throughout the study with no adjustments. The median azacitidine cycle length was 34 days (range 15 to 92). LDara-C was administrated for a median of 4.5 cycles (range 1 to 15), BSC only patients for a median of 7 cycles (range 1 to 26, 6.2 months), and intensive chemotherapy for 1 cycle (range 1 to 3, i.e. induction plus 1 or 2 consolidation cycles, with cytarabine and anthracycline). Median follow-up for the overall survival analysis was 21.1 months. Overall analysis (ITT): AZA (N=179 vs. CCR (N=179). Analysis by CCR treatment selection: AZA (N=117) vs. BSC (n=105); AZA (N=45) vs. LD Ara-C (N=49); AZA (N=17) vs. Intensive Chemo (N=25). Four patients in the azacitidine group and 14 in the CCR group never received but were followed for overall survival and were included in the ITT analysis. Eight patients went on to transplant after treatment (4 in the azacitidine group and 4 in the CCR group: BSC [n=2], LDara-C [n=1], intensive chemotherapy [n=1]) and were also included in the ITT analysis.

Overall Survival

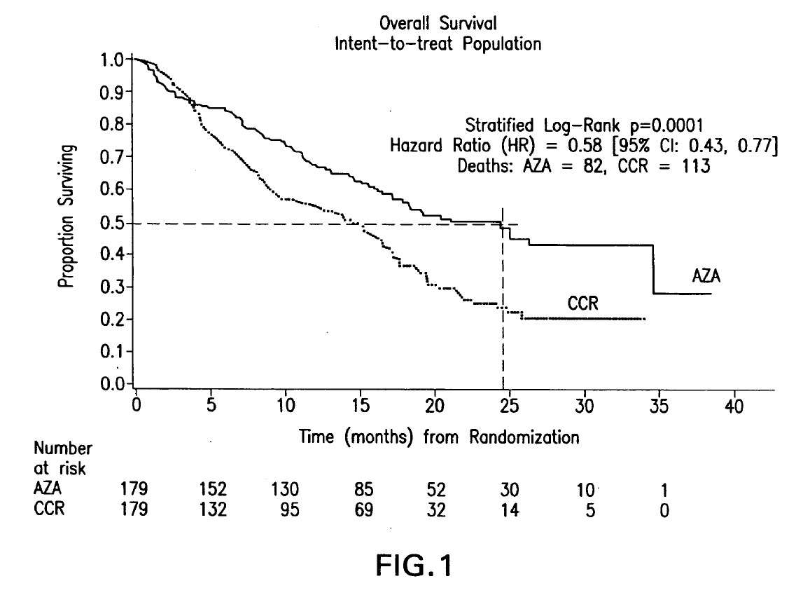

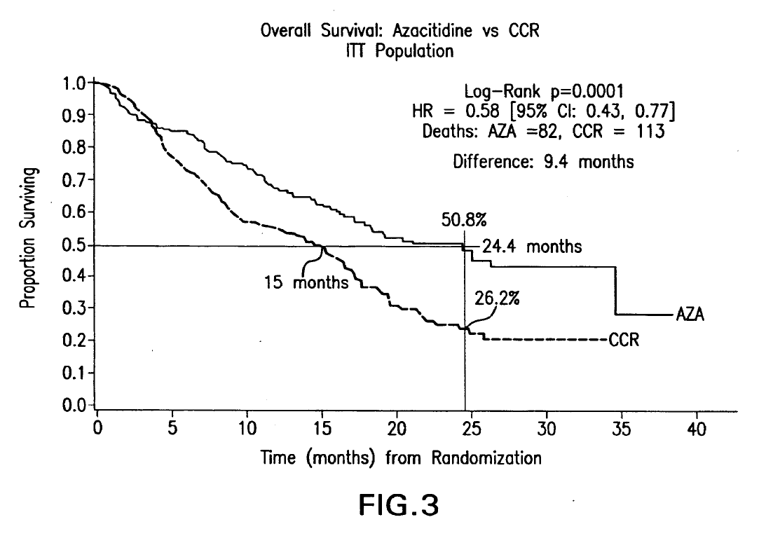

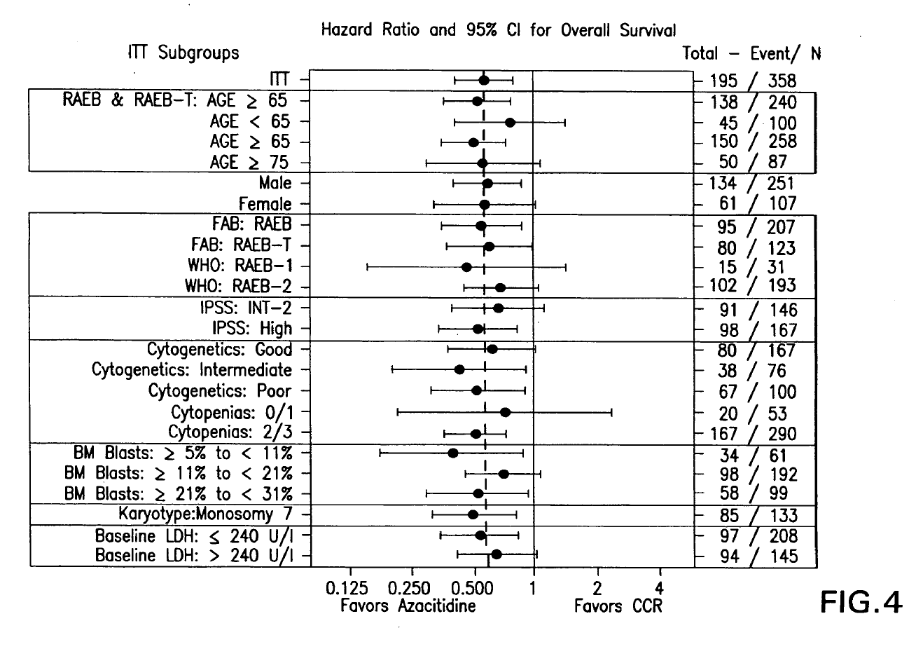

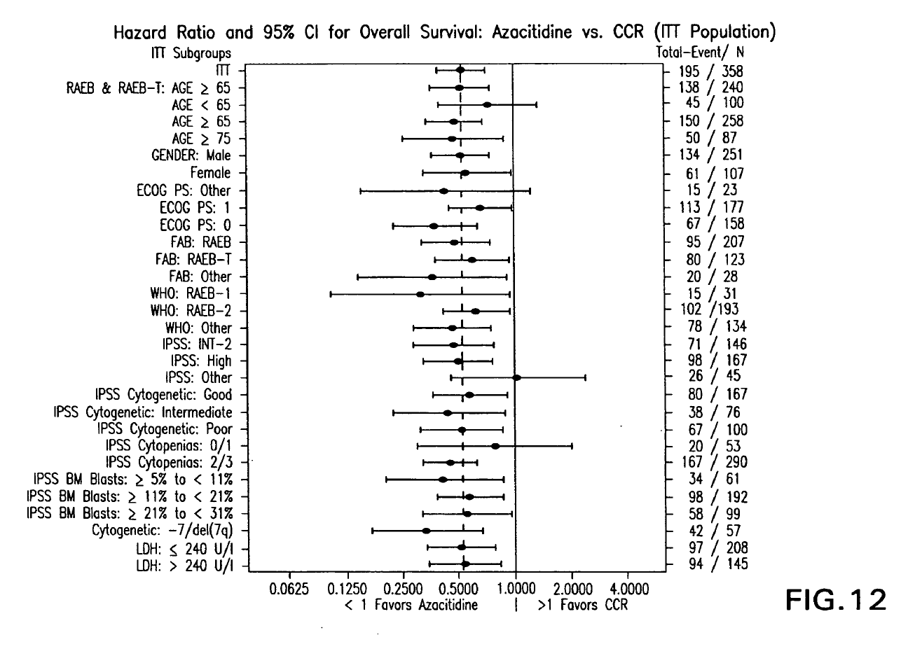

Azacitidine demonstrated statistically superior overall survival vs. conventional care regimens. After a median follow-up of 21.1 months (range 0 to 38.4), median Kaplan-Meier overall survival was 24.4 months in the azacitidine group compared with 15 months in the CCR group, for a difference of 9.4 months (stratified log-rank p=0.0001) (FIGS. 1 and 3). The hazard ratio (Cox Model) was 0.58 (95% CI: 0.43-0.77) indicating a 42% reduction in risk of death in the azacitidine group and a 74% overall survival advantage (FIGS. 4 and 12). At two year, 50.8% (95% CI: 42.1-58.8) of patients in the azacitidine group were alive compared with 26% (95% CI: 18.7-34.3) in the CCR group (p<0.0001). After approximately 100 days (about 3 months), with 78% (140/179) of azacitidine patients completing 3 cycles of therapy, the Kaplan-Meier curves for the azacitidine and CCR groups separated for the remainder of the trial.

Results in the predefined patient subgroups (based on age, gender, FAB classification, IPSS, WHO classification, karyotype, and LDH) also showed a consistent overall survival benefit for the azacitidine group (FIGS. 1 and 3). In particular, IPSS cytogenetic subgroups showed significant overall survival differences favoring the azacitidine group versus the CCR group (hazard ratio; log-rank p): Poor, 11.2 months (0.52, p=0.011); Intermediate, 9.3 months (0.43, p=0.017); and Good, median not reached (0.62, log-rank p=0.038). In patients with −7/del(7q), median Kaplan-Meier overall survival was 13.1 months (95% CI, 9.9 to 24.5) in the azacitidine group (n=30) compared with 4.6 months (95% CI, 3.5 to 6.7) in the CCR group (n=27) (stratified log-rank p=0.002, hazard ratio, 0.33 (95% CI, 0.16 to 0.68). Additionally, sensitivity analyses exploring the influence of the 8 transplanted patients included in the ITT analyses above did not influence the significance of the overall survival results for azacitidine.

The survival benefits of azacitidine were consistent regardless of the CCR treatment options. Differences in median overall survival (hazard ratio; log-rank p) between the azacitidine subgroups and the CCR subgroups of BSC, LDara-C, and intensive chemotherapy were 9.6 months (0.58; p=0.005), 9.2 months (0.36; p=0.0006), and 9.4 months (0.76, 95% CI: 0.33 to 1.74), respectively (Table 3A). Similar to the primary overall survival comparison (azacitidine vs. CCR), results from the investigator pre-selection subgroup analysis of overall survival showed significant differences between azacitidine (n=117) and BSC (n=105) (p=0.005) and azacitidine (n=45) and LDara-C (n=49) (p=0.0006). The difference in the comparison between azacitidine (n=17) and intensive chemotherapy (n=25), however, was not significant (0.51) (Table 3A).

The significant prolongation of overall survival observed with azacitidine compared with CCR was not dependent on the achievement of complete remission (HR=0.39 [95% CI: 0.14-1.15], log rank p=0.078). The achievement of hematologic improvement, partial remission, or complete remission contributed to but was not required for improvement in overall survival with azacitidine treatment.

To date, azacitidine is the only agent to demonstrate survival benefit in MDS compared to conventional care regimens, and the only epigenetic modifier to show survival benefits in cancer. The study described herein represented the largest study ever conducted in higher risk MDS. These results, showing a significant improvement in survival in the most advanced MDS patients, demonstrated the benefit azacitidine can provide to treat the disease. Building on the established data from earlier clinical studies, which showed that azacitidine offers transfusion independence benefits to patients with MDS to improve the overall quality of life, the present study showed that azacitidine not only improves patient's life, but extends it as well.

Secondary Efficacy Endpoints

Red blood cell transfusion independence, hematologic remission, and hematologic improvement were also significantly increased with azacitidine as compared with combined conventional care regimens. Azacitidine was well tolerated.

Time to AML Transformation

Assessed over the entire trial, median time to transformation to AML or death was 13.0 months (95% CI: 9.9-15.0) in the azacitidine group compared with 7.6 months (95% CI: 5.4-9.8) in the CCR group (hazard ratio: 0.68, log-rank p<0.003).

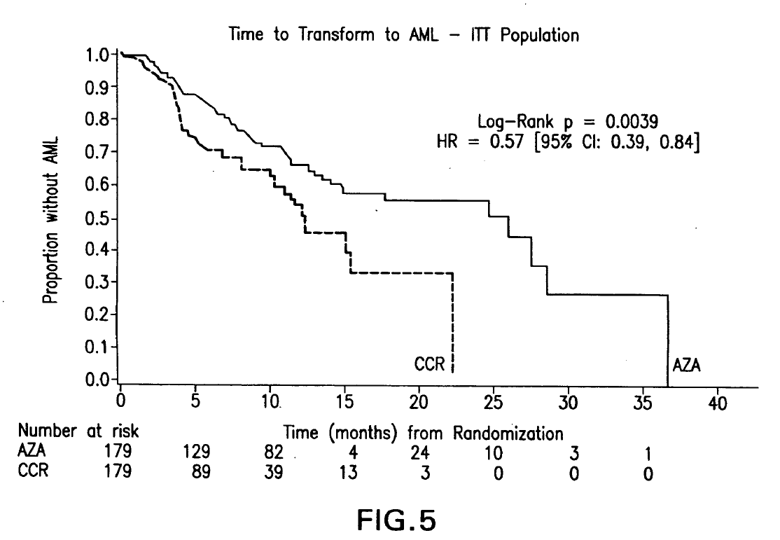

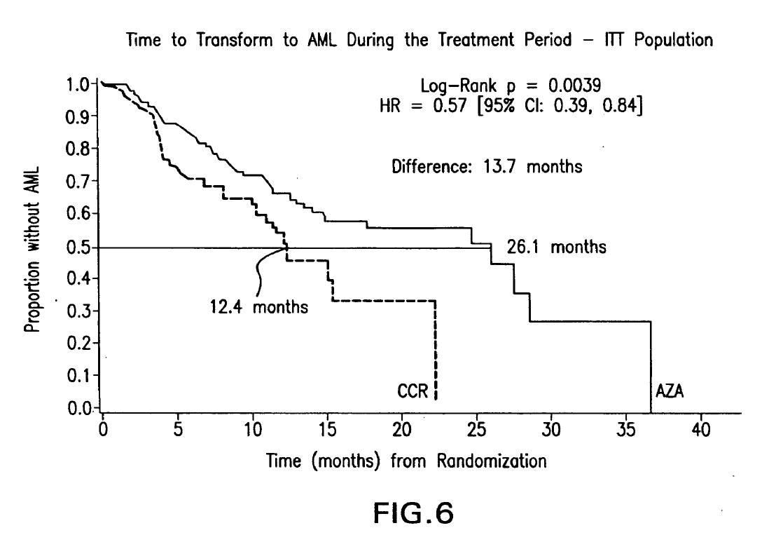

Time to AML transformation was assessed during treatment with a median of 26.1 months (95% CI: 15.0-28.7) in the azacitidine group compared with 12.4 months (95% CI: 10.4-15.4) in the CCR group (log-rank p=0.004, FIGS. 5 and 6).

Median time to AML transformation was 17.8 months (95% CI, 13.6 to 23.6) in the azacitidine group compared with 11.5 months (95% CI, 8.3 to 14.5) in the CCR group (hazard ratio, 0.50 (95% CI, 0.35 to 0.70), log rank p<0.0001).

Hematologic Response and Improvement Rates

Complete and partial remission rates were significantly higher in the azacitidine group than in the CCR group (Table 3B). Using the investigator pre-selection analysis, remission rates were generally significantly higher with azacitidine compared with either BSC or LDara-C, but no significant differences in remission rates were observed when comparing azacitidine with intensive chemotherapy (Table 3C). Time to disease progression, relapse after complete or partial remission, or death was significantly longer in the azacitidine group (median, 14.1 months) than in the CCR group (median, 8.8 months, log-rank P=0.047). Erythroid and platelet improvement rates were significantly higher in the azacitidine group compared with the CCR group (Table 3B). Major erythroid improvement was observed in 39.5% (62 of 157) vs. 10.6% (17 of 160) of patients in the azacitidine vs. CCR groups, respectively, (p<0.0001). Major platelet improvement was observed in 32.6% (46 of 141) vs. 14% (18 of 129) of patients in the azacitidine vs. CCR groups, respectively (p=0.0003). No significant differences for major neutrophil improvement were observed between groups. Duration of hematologic improvement was significantly longer in the azacitidine group (median, 13.6 months, 95% CI, 10.1 to 16.3) than in the CCR group (median, 5.2 months, 95% CI, 4.1 to 9.7, P=0.0002). 50 of 111 (45%, 95% CI, 35.6 to 54.8) baseline RBC transfusion-dependent patients in the azacitidine group became transfusion independent compared with 13 of 114 (11.4%, 95% CI, 6.2 to 18.7) in the CCR group (P<0.0001).

Overall, 51 of 179 (28.5%) patients in the azacitidine group achieved complete+partial remission compared with 21 of 179 (11.7%, p=0.0001) in the CCR group, including 5 of 105 (5%), 6 of 49 (12.2%), and 10 of 25 (40%) in the BSC, LDara-C, and intensive chemotherapy subgroups, respectively. 17% (30 of 179) and 8% (14 of 179) of patients in the azacitidine and CCR groups, respectively, had a complete remission (p=0.02). The proportion of patients showing any hematologic improvement was significantly higher in the azacitidine group (87 of 177, 49.2%) compared with the CCR group (51 of 178, 28.7%, p<0.0001).

Transfusion Independence

45% (95% CI: 35.6-54.8) of patients in the azacitidine group became RBC transfusion independent after being baseline dependent compared with 11.4% (95% CI: 6.2-18.7) in the CCR group (p=0.0001). The effect on platelet transfusions showed no significant differences between the azacitidine and CCR groups, which was likely due to the small numbers of patients with baseline platelet transfusion dependence in the azacitidine (n=38) and CCR (n=27) groups.

Infections Requiring Intravenous Antimicrobials

The rate of infections requiring intravenous antimicrobials per patient year in the azacitidine group was 0.60 (95% CI, 0.49 to 0.73) compared with 0.92 (95% CI, 0.74 to 1.13) in the CCR group, indicating a 34% reduction (hazard ratio, 0.66, 95% CI, 0.49 to 0.87, P=0.003). Using the investigator pre-selection analysis, per patient year rates were similar when comparing azacitidine (0.66) and BSC (0.61) (hazard ratio: 1.1, 95% CI, 0.74 to 1.65, P=0.68), but significantly lower with azacitidine (0.44) compared with LDara-C (1.00) (hazard ratio: 0.44, 95% CI, 0.25 to 0.86, P=0.017) or with azacitidine (0.64) versus intensive chemotherapy (2.30) (hazard ratio: 0.28, 95% CI, 0.13 to 0.60, P=0.0006).

Safety

Discontinuations prior to study closure due to adverse events were observed in 12.6% of patients in the azacitidine group compared with (7.3%) in the CCR group. The 2 active therapies in the CCR group showed similar rates with azacitidine but BSC had a much lower rate of discontinuations due to adverse events (3.9%). The most frequently observed treatment-related adverse events (including Grade 3-4 events) were peripheral blood cytopenias, frequently observed across all treatments, which led to discontinuation prior to study closure in 4.6% in the azacitidine group and 2.4% in the CCR group. The most common treatment-related non-hematologic adverse events included injection site reactions with azacitidine, and nausea, vomiting, fatigue, and diarrhea with azacitidine, LDara-C, and intensive chemotherapy (Table 3D and 3E). During the first 3 cycles of treatment, deaths occurred in 14 (8%) of patients in the azacitidine group and 25 (14%) in the CCR group. The most common causes of death in either group were related to underlying disease, thrombocytopenia, sepsis/infection, hemorrhage, and respiratory complications. Transformation to AML was also a cause of death during the first 3 cycles of treatment but observed only in the CCR group. Deaths considered to be related to treatment during the first 3 cycles were observed in 4 patients in the azacitidine group (septic shock, cerebral hemorrhage, hematemesis, respiratory tract infection) and 1 patient in the CCR group (receiving LDara-C) (cerebral ischemia).

In the higher risk MDS population, the most frequently observed treatment-related adverse events (including Grade 3 and 4 events) were blood cytopenias, frequently observed across all treatments, which led to early withdrawal in 4.6%, 4.5%, and 2% of patients in the azacitidine, LDara-C, and BSC treatment groups, respectively (Table 3F).

The most common treatment-related non-hematologic adverse events included injection site reactions with azacitidine, and nausea, vomiting, fatigue, and diarrhea across the azacitidine, low-dose ara-C, and intensive chemotherapy treatment groups. During treatment and follow-up, deaths were reported in 45% of patients in the azacitidine group, and 62%, 59%, and 79% of patients, respectively, in the BSC, LDara-C, and intensive chemotherapy subgroups. The major causes of death were infection and AML (>30% blasts).

Discussion:

Results of the phase III, randomized, controlled comparative trial showed that azacitidine was the first drug treatment to prolong overall survival in higher risk MDS patients. While allogeneic stem cell transplantation is potentially curative in MDS, its use is limited by older age, a lack of donors, and increased transplant-related mortality. In a previous randomized phase III CALGB trial comparing azacitidine with BSC (See, e.g., J. Clin. Oncol. 2002, 20(10):2429-40), the azacitidine group showed a trend for improved overall survival over BSC. The finding was possibly limited by a heterogeneous patient population and a cross-over trial design, with 51% of BSC patients subsequently receiving azacitidine. Findings of the CALGB trial were also lessened by the use of BSC, a treatment not considered as intensive care in higher risk MDS by many clinicians.

No crossover was allowed in the present study. The present study included only patients with higher risk MDS. Additionally, the study compared azacytidine to three frequently used treatments (LDara-C, intensive chemotherapy, or BSC) for higher risk MDS including two active therapties (LDara-C, or intensive chemotherapy). As there is no current consensus on the use of those three regimens, their allocation for patients was made by the investigators based on patient age, general condition, presence of co-morbidities, and personal choice.

Overall survival in the present study showed an advantage of 9.4 months for the azacitidine group over the CCR group, corresponding to a 42% reduction in risk of death. The robustness of this overall survival benefit was further shown in the nearly 2-fold higher proportion of patients in the azacitidine group surviving at two years compared with those in the CCR group. This overall survival advantage with azacitidine in the primary, ITT analysis was highly similar to that seen using the secondary, investigator-selection analysis with median survival differences ranging from 9.2 months to 9.6 months between azacitidine and the three CCR subgroups.

The onset of the significant survival benefit occurred early in the present study with the Kaplan Meier curves for the azacitidine and CCR groups separating permanently at approximately 3 months with nearly 80% of patients in the azacitidine group having completed more than three cycles of treatment. Results obtained in the subgroup analyses for age, gender, FAB and WHO classification, karyotype; and LDH confirmed the robustness of the overall survival results achieved in the ITT population. The survival advantage in the azacitidine group was maintained irrespective of IPSS cytogenetic risk group (favorable, intermediate, and poor), an important finding as abnormal karyotype is a frequent finding in MDS and a strong prognostic factor for a poorer outcome.

Findings in the secondary efficacy endpoints support the overall survival advantage demonstrated in the azacitidine group. Azacitidine treatment significantly prolonged the time to AML transformation or death and the time to transformation to AML compared with CCR. Significantly higher IWG-defined response rates were observed in the azacitidine group compared with the CCR group, including complete or partial remission and major erythroid hematologic improvement. The superior response rates observed in the azacitidine group were driven by notably lower rates in the LDara-C and BSC subgroups. Response rates in the small intensive chemotherapy subgroup were higher than those seen in the azacitidine group. Remission and hematologic improvement rates also endured longer in the azacitidine group than the CCR group.

RBC transfusion independence after baseline dependence was significantly higher in the azacitidine group than in the CCR group, an important finding as transfusion dependency had been shown to be an significant marker of poorer outcome in MDS. No differences were observed between the azacitidine and CCR group for platelet transfusion independence, which was likely due to the small number of patients with baseline dependency. Additionally, although azacitidine treatment was not associated with an increase in the proportion of patients with neutrophil improvement compared with the CCR group, a 33% reduction in the risk of infection requiring intravenous antimicrobials was observed in the azacitidine group.

Grade 3 and 4 neutropenia was observed more frequently in the azacitidine group than in the BSC subgroup, and at a similar rate compared with the LDara-C or intensive chemotherapy subgroups. Thrombocytopenia was also observed more commonly with azacitidine than with BSC but less frequently than with LDara-C and intensive chemotherapy. However, despite the higher frequency of thrombocytopenia and neutropenia observed with azacitidine compared with BSC, the overall occurrence of bleeding and infection was similar in both treatments.

Nonhematologic adverse events more commonly reported in the azacitidine group than with the BSC subgroup, such as injection site reactions, nausea, and vomiting, were largely Grade 1-2 in severity, were well recognized events observed with azacitidine treatment, and caused no patients to discontinue therapy. Generally, injection site reactions were easily managed by varying injection sites and by applying a post-injection cool or warm compress for 15 minutes.

The results demonstrated the first finding of an overall survival benefit in the treatment of MDS. Significantly longer overall survival was clearly shown with azacitidine treatment compared with the CCR group, which comprised three other commonly used treatments in patients with higher risk MDS. The overall survival advantage was demonstrated irrespective of the CCR regimen (BSC, LDara-C, or intensive chemotherapy) and regardless of a good, intermediate, or poor IPSS cytogenetic risk. The results showing the overall survival benefit demonstrated with azacitidine, given for a median of 9 cycles, was supported by a significant prolongation in time to AML transformation as well as increases in transfusion independence, complete and partial remissions, and major hematologic improvements. The significant increases in transfusion independence and hematologic improvement particularly suggested that decreasing cytopenias reduces the risk of their lethal complications, thus altering the natural disease course of MDS. These findings strongly established azacitidine as the reference treatment in higher risk MDS, against which newer treatments will have to be compared or combined with in future trials in these patients.

TABLE 2A
Baseline Demographics

| | | | Conventional Care Regimens | | | | | | | |
|---|---|---|---|---|---|---|---|---|---|---|
| | | | | | | | Intensive | | | |
| | Azacitidine | | BSC Only | | LDAC, | | Chemo | | CCR Total | |
| Parameter | N = 179 | | N = 105 | | n = 49 | | N = 25 | | N = 179 | |
| Age (years) | | | | | | | | | | |
| N | 179 | | 105 | | 49 | | 25 | | 179 | |
| Median | 69.0 | | 70.0 | | 71.0 | | 65.0 | | 70.0 | |
| Min, Max | 42, 83 | | 50, 88 | | 56, 85 | | 38, 76 | | 38, 88 | |
| ≦64, n (%) | 57 | (31.9) | 24 | (22.9) | 7 | (14.3) | 12 | (48.0) | 43 | (24) |
| ≧65, n (%) | 122 | (68.1) | 81 | (77.1) | 42 | (85.7) | 13 | (52.0) | 136 | (76) |
| Gender - n (%) | | | | | | | | | | |
| Male | 132 | (73.7) | 67 | (63.8) | 35 | (71.4) | 17 | (68.0) | 119 | (66.5) |
| Female | 47 | (26.3) | 38 | (36.2) | 14 | (28.6) | 8 | (32.0) | 60 | (33.5) |
| FAB Classification* - | | | | | | | | | | |
| n (%) | | | | | | | | | | |
| RAEB | 104 | (58.1) | 68 | (64.8) | 25 | (51.0) | 10 | (40.0) | 103 | (57.5) |
| RAEB-T | 61 | (34.1) | 30 | (28.6) | 19 | (38.8) | 13 | (52.0) | 62 | (34.6) |
| CMML | 6 | (3.3) | 4 | (3.8) | 1 | (2.0) | 0 | | 5 | (2.8) |
| AML | 1 | (0.6) | 0 | | 0 | | 1 | (4.0) | 1 | (0.6) |
| IPSS - n (%)^{†} | | | | | | | | | | |
| Intermediate-1 | 5 | (2.8) | 9 | (8.6) | 2 | (4.1) | 2 | (8.0) | 13 | (7.3) |
| Intermediate-2 | 76 | (42.5) | 46 | (43.8) | 21 | (42.9) | 3 | (12.0) | 70 | (39.1) |
| High | 82 | (45.8) | 46 | (43.8) | 21 | (42.9) | 18 | (72.0) | 85 | (47.5) |
| Karyotype - n (%) | | | | | | | | | | |
| Good | 83 | (46) | 47 | (45) | 28 | (57) | 9 | (36) | 84 | (47) |
| Intermediate | 37 | (21) | 23 | (22) | 12 | (25) | 4 | (16) | 39 | (22) |
| Poor | 50 | (28) | 31 | (29) | 8 | (16) | 11 | (44) | 50 | (28) |
| Missing | 9 | (5) | 4 | (4) | 1 | (2) | 1 | (4) | 6 | (3) |
| WHO Classification - | | | | | | | | | | |
| n (%) | | | | | | | | | | |
| RAEB-1 | 14 | (7.8) | 13 | (12.4) | 3 | (6.1) | 1 | (4.0) | 17 | (9.5) |
| RAEB-2 | 98 | (54.7) | 60 | (57.1) | 24 | (49.0) | 11 | (44.0) | 95 | (53.1) |
| CMMoL-1 | 1 | (0.3) | 0 | | 0 | | 0 | | 0 | |
| CMMoL-2 | 10 | (5.6) | 3 | (2.9) | 0 | | 2 | (8.0) | 5 | (2.8) |
| AML | 55 | (30.7) | 27 | (25.7) | 20 | (40.8) | 11 | (44.0) | 58 | (32.4) |
| Indeterminate | 1 | (0.6) | 2 | (1.9) | 2 | (4.1) | 0 | | 4 | (2.2) |
| ECOG Performance | | | | | | | | | | |
| Status - n (%) | | | | | | | | | | |
| 0 | 78 | (43.6) | 36 | (34.3) | 29 | (59.2) | 15 | (60.0) | 80 | (44.7) |
| 1 | 86 | (48.0) | 59 | (56.2) | 17 | (34.7) | 10 | (40.0) | 86 | (48.0) |
| 2 | 13 | (7.3) | 8 | (7.6) | 2 | (94.1) | 0 | | 10 | (5.6) |
| Missing | 2 | (1.1) | 2 | (1.9) | 1 | (2.0) | 0 | | 3 | (1.7) |
| Time Since Original | | | | | | | | | | |
| Diagnosis (years) - | | | | | | | | | | |
| n (%) | | | | | | | | | | |
| <1 year | 92 | (51.4) | 53 | (50.5) | 28 | (57.1) | 14 | (56.0) | 95 | (53.1) |
| 1 to <2 years | 37 | (20.7) | 27 | (25.7) | 12 | (24.5) | 6 | (24.0) | 45 | (25.1) |
| 2 to <3 years | 20 | (11.2) | 6 | (5.7) | 3 | (6.1) | 1 | (4.0) | 10 | (5.6) |
| ≧3 years | 30 | (16.8) | 19 | (18.1) | 6 | (12.2) | 4 | (16.0) | 29 | (16.2) |

*Another 3.9% and 4.5% of patients in the azacitidine and CCR groups, respectively, had myeloproliferative disease or were disease was indeterminate.
^{†}Another 8.9% and 6.2% of patients in the azacitidine and CCR groups, respectively, had disease not applicable to IPSS or were indeterminate

TABLE 2B
Baseline Demographics by Investigator Pre-Selection

| Investigator Pre-Selection | | | | | |
|---|---|---|---|---|---|
| | | | | Intensive | |
| BSC Only | | LDara-C | | Chemotherapy (IC) | |
| (n = 222) | | (n = 94) | | (n = 42) | |
| Randomization | | | | | |
| Azacitidine | BSC | Azacitidine | LDara-C | Azacitidine | IC |
| N = 117 | N = 105 | N = 45 | N = 49 | N = 17 | N = 25 |

Parameter

| Age (years) | | | | | | | | | | | | |
|---|---|---|---|---|---|---|---|---|---|---|---|---|
| N | 117 | | 105 | | 45 | | 49 | | 17 | | 25 | |
| Median | 69.0 | | 70.0 | | 69.0 | | 71.0 | | 63.0 | | 65.0 | |
| Min, Max | 52, 83 | | 50, 88 | | 42, 92 | | 56, 85 | | 45, 79 | | 38, 76 | |
| ≦64, n (%) | 33 | (28.2) | 24 | (22.9) | 14 | (31.1) | 7 | (14.3) | 10 | (58.8) | 12 | (48.0) |
| ≧65, n (%) | 84 | (71.8) | 81 | (77.1) | 31 | (68.9) | 42 | (85.7) | 7 | (41.2) | 13 | (52.0) |
| Gender - n (%) | | | | | | | | | | | | |
| Male | 81 | (69.2) | 67 | (63.8) | 39 | (86.7) | 35 | (71.4) | 12 | (70.6) | 17 | (68.0) |
| Female | 36 | (30.8) | 38 | (36.2) | 6 | (13.3) | 14 | (29.6) | 5 | (29.4) | 9 | (32.0) |
| FAB Classification - | 69 | (59.0) | 68 | (64.8) | 27 | (60.0) | 25 | (51.0) | 8 | (47.1) | 10 | (40.0) |
| n (%) RAEB | | | | | | | | | | | | |
| RAEB-T | 38 | (32.5) | 30 | (28.6) | 15 | (33.3) | 19 | (38.8) | 8 | (47.1) | 13 | (52.0) |
| CMML | 5 | (4.3) | 4 | (3.8) | 1 | (2.2) | 1 | (2.0) | 0 | (0.0) | 0 | (0.0) |
| AML | 0 | (0.0) | 0 | (0.0) | 1 | (2.2) | 0 | (0.0) | 0 | (0.0) | 1 | (4.0) |
| IPSS - n (%) | 4 | (3.4) | 9 | (8.6) | 1 | (2.2) | 2 | (4.1) | 0 | (0.0) | 2 | (8.0) |
| Intermediate-1 | | | | | | | | | | | | |
| Intermediate-2 | 48 | (41.0) | 46 | (43.8) | 22 | (48.9) | 21 | (42.9) | 6 | (35.3) | 3 | (12.0) |
| High | 57 | (48.7) | 46 | (43.8) | 19 | (42.2) | 21 | (42.9) | 6 | (35.3) | 18 | (72.0) |
| Karyotype - n (%) Good | 53 | (45.3) | 47 | (44.8) | 24 | (53.3) | 28 | (57.1) | 6 | (35.3) | 9 | (36.0) |
| Intermediate | 25 | (21.4) | 23 | (21.9) | 7 | (15.6) | 12 | (24.5) | 5 | (29.4) | 4 | (16.0) |
| Poor | 33 | (28.2) | 31 | (29.5) | 13 | (28.9) | 8 | (16.3) | 4 | (23.5) | 11 | (44.0) |
| Missing | 6 | (5.1) | 4 | (3.8) | 1 | (2.2) | 1 | (2.0) | 2 | (11.8) | 1 | (4.0) |
| WHO Classification - | | | | | | | | | | | | |
| 1/n (%) | | | | | | | | | | | | |
| RAEB-1 | 8 | (6.8) | 13 | (12.4) | 3 | (6.7) | 3 | (6.1) | 3 | (17.6) | 1 | (4.0) |
| RAEB-2 | 63 | (53.8) | 60 | (57.1) | 27 | (60.0) | 24 | (49.0) | 8 | (47.1) | 11 | (44.0) |
| CMML-1 | 1 | (0.9) | 0 | (0.0) | 0 | (0.0) | 0 | (0.0) | 0 | (0.0) | 0 | (0.0) |
| CMML-2 | 8 | (6.8) | 3 | (2.9) | 1 | (2.2) | 0 | (0.0) | 1 | (5.9) | 2 | (8.0) |
| AML | 36 | (30.8) | 27 | (25.7) | 14 | (31.1) | 20 | (40.8) | 5 | (29.4) | 11 | (44.0) |
| Indeterminate | 1 | (0.9) | 2 | (1.9) | 0 | (0.0) | 2 | (4.1) | 0 | (0.0) | 0 | (0.0) |
| ECOG Performance | | | | | | | | | | | | |
| Status - n (%) | | | | | | | | | | | | |
| 0 | 47 | (40.2) | 36 | (34.3) | 21 | (46.7) | 29 | (59.2) | 10 | (58.8) | 15 | (60.0) |
| 1 | 59 | (50.4) | 59 | (56.2) | 21 | (46.7) | 17 | (34.7) | 6 | (35.3) | 10 | (40.0) |
| 2 | 11 | (9.4) | 8 | (7.6) | 1 | (2.2) | 2 | (4.1) | 1 | (5.9) | 0 | (0.0) |
| Missing | 0 | (0.0) | 2 | (1.9) | 2 | (4.4) | 1 | (2.0) | 0 | (0.0) | 0 | (0.0) |
| Time Since Original | | | | | | | | | | | | |
| Diagnosis (years) - | | | | | | | | | | | | |
| n (%) | | | | | | | | | | | | |
| <1 year | 53 | (45.3) | 53 | (50.5) | 29 | (64.4) | 28 | (57.1) | 10 | (58.8) | 14 | (56.0) |
| 1 to <2 years | 29 | (24.8) | 27 | (25.7) | 7 | (15.6) | 12 | (24.5) | 1 | (5.9) | 6 | (24.0) |
| 2-<3 years | 14 | (12.0) | 6 | (5.7) | 4 | (8.9) | 3 | (6.1) | 2 | (11.8) | 1 | (4.0) |
| ≧3 years | 21 | (17.9) | 19 | (18.1) | 5 | (11.1) | 6 | (12.2) | 4 | (23.5) | 4 | (16.0) |

TABLE 3A
Kaplan-Meier Median Overall Survival Comparison per Investigator Pre-Selection.

| | Investigator Pre-Selection | | | | | | | | |
|---|---|---|---|---|---|---|---|---|---|
| | BSC Only | | | LDara-C | | | IC* | | |
| | (n = 222) | | | (n = 94) | | | (n = 42) | | |
| | Randomization | | | | | | | | |
| | AZA | BSC | | AZA | LDara-C | | AZA | | |
| | N = 117 | N = 105 | Difference | N = 45 | N = 49 | Difference | N = 17 | IC N = 25 | Difference |
| Overall Survival, months | 21.1 | 11.5 | 9.6 | 24.5 | 15.3 | 9.2 | 25.1 | 15.7 | 9.4 |

| | | | |
|---|---|---|---|
| HR* (95% CI), p value† | 0.58 (0.40 to 0.85), 0.005 | 0.36 (0.20 to 0.65), 0.0006 | 0.76 (95% CI: 0.33 to 1.74) |

*Abbreviations: IC = intensive chemotherapy; HR = hazard ratio
†From stratified Cox proportional hazards model adjusted for treatment, subgroup, ECOG performance status, LDH, hemoglobin, number of RBC transfusions, and presence or absence of cytogenetic −7/del(7q) abnormality. All subgroup-by-treatment interactions were not significant (p > 0.20).

TABLE 3B
Hematologic Response and Improvement Rates, n/N (%)*

| | Azacitidine | CCR Total | |
|---|---|---|---|
| Parameter | (N = 179) | (N = 179) | P value^{†} |
| Hematologic Response | | | |
| Overall (CR + PR) | 51 (28.5) | 21 (11.7) | 0.0001 |
| Complete Remission | 30/179 (16.8) | 14/179 (7.8) | 0.0150 |
| Partial Remission | 21/179 (11.7) | 7/179 (3.9) | 0.0094 |
| Stable Disease | 75/179 (41.9) | 65/179 (36.3) | 0.3297 |
| Hematologic Improvement^{‡} | | | |
| Any Improvement | 87/177 (49.2) | 51/178 (28.7) | <0.0001 |
| (Major + Minor), n/N (%) | | | |
| HI-E Major, n/N (%) | 62/157 (39.5) | 17/160 (10.6) | <0.0001 |
| HI-P Major, n/N (%) | 46/141 (32.6) | 18/129 (14.0) | 0.0003 |
| HI-N Major, n/N (%) | 25/131 (19.1) | 20/111 (18.0) | 0.8695 |

*Hematologic response and improvement based on IWG 2000 criteria for MDS.
^{†}P-value from Fisher's exact test for comparing the response rates between the Azacitidine group and the combined group of conventional care regimens.
^{‡}HI-E = Erythroid Improvement; HI-P = Platelet Improvement; HI-N = Neutrophil Improvement.

TABLE 3C
Hematologic Response by Investigator Pre-Selection.

| Investigator Pre-Selection | | |
|---|---|---|
| BSC Only | LDara-C | Intensive Chemotherapy (IC) |
| (n = 222) | (n = 94) | (n = 42) |

| Randomization | | | | | | | | |
|---|---|---|---|---|---|---|---|---|
| AZA | BSC | | AZA | LDara-C | | AZA | IC | |
| N = 117 | N = 105 | P-Value | N = 45 | N = 49 | P-Value | N = 17 | N = 25 | P-Value |

Parameter

| | | | | | | | | | | | | | | | |
|---|---|---|---|---|---|---|---|---|---|---|---|---|---|---|---|
| Overall | 32 | (27.4) | 5 | (4.8) | <0.0001 | 14 | (31.1) | 6 | (12.2) | 0.042 | 5 | (29.4) | 10 | (40.0) | 0.531 |
| (CR + PR) | | | | | | | | | | | | | | | |
| Complete | 14 | (12.0) | 1 | (1.0) | 0.0008 | 11 | (24.4) | 4 | (8.2) | 0.0471 | 5 | (29.4) | 9 | (36.0) | 0.7468 |
| Remission | | | | | | | | | | | | | | | |
| (CR) | | | | | | | | | | | | | | | |
| Partial | 18 | (15.4) | 4 | (3.8) | 0.0058 | 3 | (6.7) | 2 | (4.1) | 0.6677 | 0 | (0.0) | 1 | (4.0) | 1.0000 |
| Remission (PR) | | | | | | | | | | | | | | | |
| Stable | 52 | (44.4) | 41 | (39.0) | 0.4959 | 15 | (33.3) | 18 | (36.7) | 0.8297 | 8 | (47.1) | 6 | (24.0) | 0.1836 |
| Disease (SD) | | | | | | | | | | | | | | | |

IWG Hematologic Improvement

| | | | | | | | | | | | | | | | |
|---|---|---|---|---|---|---|---|---|---|---|---|---|---|---|---|
| Any | 57/115 | (49.6) | 32/105 | (30.5) | 0.0058 | 24/45 | (53.3) | 12/48 | (25.0) | 0.0061 | 6/17 | (35.3) | 7/25 | (28.0) | 0.7377 |
| Improvement, | | | | | | | | | | | | | | | |
| n/N (%) | | | | | | | | | | | | | | | |
| HI-E Major, | 39/100 | (39.0) | 8/96 | (8.3) | <0.0001 | 19/43 | (44.2) | 4/14 | (9.8) | 0.0005 | 4/14 | (28.6) | 5/23 | (21.7) | 0.7046 |
| n/N (%) | | | | | | | | | | | | | | | |
| HI-P Major, | 27/89 | (30.3) | 8/96 | (10.3) | 0.0020 | 14/37 | (37.8) | 6/31 | (19.4) | 0.1153 | 5/15 | (33.3) | 4/20 | (20.0) | 0.4505 |
| n/N (%) | | | | | | | | | | | | | | | |
| HI-N Major, | 13/85 | (15.3) | 13/66 | (19.7) | 0.5193 | 9/33 | (27.3) | 3/28 | (10.7) | 0.1220 | 3/13 | (23.1) | 4/17 | (23.5) | 1.0000 |
| n/N (%) | | | | | | | | | | | | | | | |

TABLE 3D
Reasons for Early Discontinuation and Grade 3-4 Hematologic
Toxicity.

| | AZA | CCR Total |
|---|---|---|
| | N = 179* | N = 179* |
| Deaths, overall, n (%) | 82 (46) | 113 (63) |
| Deaths during first 3 cycles | 14 (8) | 25 (14) |
| of treatment, n (%) | | |
| | N = 175^{‡} | N = 165^{‡} |

Grade 3-4 Toxicity, n (%)

| | | |
|---|---|---|
| Neutropenia | 159 (91) | 126 (76) |
| Thrombocytopenia | 149 (85) | 132 (80) |
| Anemia | 100 (57) | 112 (68) |

Patients with Baseline Grade 0-2 Shifting to Grade 3-4
during Treatment, n/N (%)

| | | |
|---|---|---|
| Neutropenia | 67/80 (84) | 46/76 (61) |
| Thrombocytopenia | 72/97 (74) | 68/94 (72) |
| Anemia | 84/156 (54) | 83/130 (64) |

*ITT population.
^{†}Only the primary causes of discontinuation are shown.
^{‡}Safety population.

TABLE 3E
Reasons for Discontinuation Before Study Closure and Grade 3-4 Hematologic Toxicity by
Investigator Pre-Selection.

| | Investigator Pre-Selection | | | | | | | | | | | |
|---|---|---|---|---|---|---|---|---|---|---|---|---|
| | BSC Only | | | | LDara-C | | | | Intensive Chemo | | | |
| | Randomization | | | | | | | | | | | |
| | AZA | | BSC | | AZA | | LDara-C | | AZA | | IC | |
| | N = 117* | | N = 105* | | N = 45* | | N = 49* | | N = 17* | | N = 25* | |
| Deaths, overall n (%) | 53 | (45) | 66 | (63) | 20 | (44) | 31 | (63) | 9 | (53) | 16 | (64) |
| Deaths during first 3 cycles | 14 | (12) | 19 | (19) | 0 | (0) | 6 | (14) | 0 | (0) | 0 | (0) |
| of Treatment, n (%) | | | | | | | | | | | | |

| | | | | | |
|---|---|---|---|---|---|
| N = 114^{‡} | N = 102^{‡} | N = 45^{‡} | N = 44^{‡} | N = 16^{‡} | N = 19^{‡} |

Grade 3-4 Toxicity, n (%)

| | | | | | | | | | | | | |
|---|---|---|---|---|---|---|---|---|---|---|---|---|
| Neutropenia | 104 | (91) | 70 | (69) | 40 | (89) | 39 | (89) | 15 | (94) | 17 | (90) |
| Thrombocytopenia | 93 | (82) | 72 | (71) | 42 | (93) | 42 | (96) | 14 | (88) | 18 | (95) |
| Anemia | 62 | (54) | 67 | (66) | 29 | (64) | 34 | (77) | 9 | (56) | 11 | (58) |

Patients with Baseline Grade 0-2
Shifting to Grade 3-4 during
Treatment, n/N (%)

| | | | | | | | | | | | | |
|---|---|---|---|---|---|---|---|---|---|---|---|---|
| Neutropenia | 45/53 | (85) | 22/46 | (48) | 14/18 | (78) | 19/24 | (79) | 8/9 | (89) | 5/6 | (83) |
| Thrombocytopenia | 49/69 | (71) | 29/54 | (54) | 17/20 | (85) | 29/30 | (97) | 6/8 | (75) | 10/10 | (100) |
| Anemia | 52/103 | (51) | 48/79 | (61) | 25/40 | (63) | 28/37 | (76) | 7/13 | (54) | 7/14 | (50) |

*ITT population.
^{†}Only the primary causes of discontinuation are shown.
^{‡}Safety population.

TABLE 3F
Grade 3-4 Hematologic Toxicity

| | % of Patients | | | |
|---|---|---|---|---|
| | | Conventional Care Regimens | | |
| | AZA | BSC | LDAC | Std Chemo |
| | N = 175 | N = 102 | N = 44 | N = 19 |
| Grade 3-4 Toxicity | | | | |
| Neutropenia | 91 | 71 | 88 | 94 |
| Thrombocytopenia | 85 | 71 | 98 | 100 |
| Anemia | 56 | 67 | 76 | 61 |
| Patients with Baseline | | | | |
| Grade 0-2 | | | | |
| Shifting to Grade 3-4 on Rx | | | | |
| Neutropenia | 84 | 48 | 79 | 83 |
| Thrombocytopenia | 74 | 54 | 97 | 100 |
| Anemia | 54 | 61 | 79 | 50 |

### 6.2 Example 2

Azacitidine (Aza) is the first drug approved for treatment of MDS. Efficacy and safety of 75 mg/m²/d subcutaneously (SC) or intravenously (IV) for 7 days every 28 days has been established. Transfusion burden is a component of high and low risk MDS; reducing transfusion dependency can enhance quality of life (QOL).

The currently approved Aza regimen is 75 mg/m²/day subcutaneously (SC) or intravenously (IV) for 7 days every 28 days. Preclinical data suggested alternative dosing regimens could provide results consistent with those seen in previous studies. An alternative dosing regimen that eliminates the need for weekend dosing would be more convenient for patients and for clinicians. To this end, 3 alternative dosing regimens, administered in 28-day cycles, were selected to determine their relative effectiveness in MDS patients:

1) AZA 5-2-2: This regimen inserts a 2-day treatment break into the currently approved 7-day dosing regimen (total cumulative dose 525 mg/m² per cycle).

2) AZA 5-2-5: This regimen involves lengthier administration (two 5-day Aza courses with a 2-day treatment break in the middle) with a lower daily dose (50 mg/m²) and slightly lower cumulative dose (500 mg/m²) per cycle.

3) AZA 5: This regimen requires briefer administration (5 days) of the currently approved 75 mg/m² daily dose, resulting in an overall lower cumulative dose (375 mg/m²) per cycle.

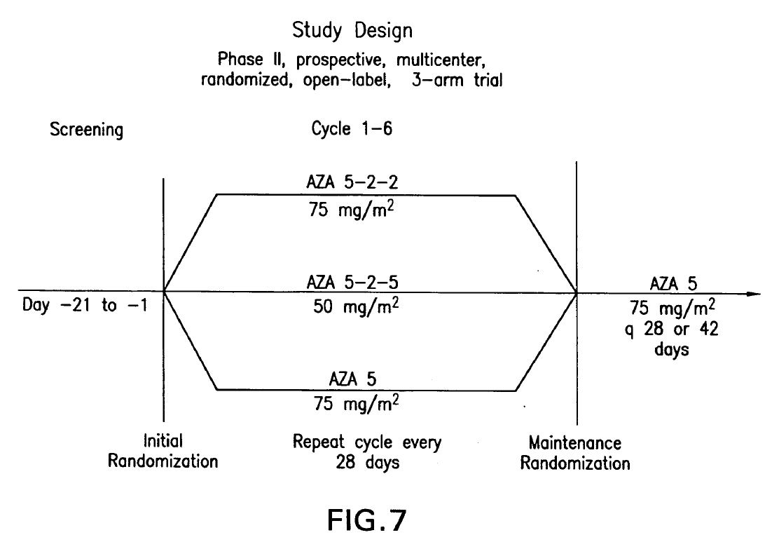

The study assessed the safety and efficacy of these 3 alternative Aza dosing strategies administered for 6 cycles. To determine whether continued therapy may improve or sustain Aza benefits, after 6 cycles patients could continue to receive Aza in a maintenance treatment phase of the study (FIG. 7).

The phase II, multi-center, randomized, open-label trial comprised 3 treatment arms (FIG. 7). Patients were randomized to 1 of 3 alternative dosing schedules, administered in 28-day cycles for 6 treatment cycles:

1) AZA 5-2-2: azacitidine 75 mg/m²/day SC×5 days, followed by 2 days of no treatment, followed by azacitidine 75 mg/m²/day SC×2 days

2) AZA 5-2-5: azacitidine 50 mg/m²/day SC×5 days, followed by 2 days of no treatment, followed by azacitidine 50 mg/m²/day SC×5 days

3) AZA 5: azacitidine 75 mg/m²/day SC×5 days.

After at least 2 cycles, Aza dose could be increased if the patient was not responding, defined as treatment failure or disease progression according to IWG 2000 criteria for MDS (≧50% increase in blasts, ≧50% decrease from maximum response levels in granulocytes or platelets, hemoglobin reduction ≧2 g/dL, or transfusion independence). Conversely, the dose could be decreased based on hematological recovery and adverse events.

Erythropoietin (EPO) was allowed in patients who were taking a stable dose of EPO for 4 weeks prior to treatment with Aza. EPO could not be started once treatment with Aza was initiated. Myeloid growth factors were allowed for treatment of neutropenic infection. Their use was stopped within 4 days of resolution of the febrile episode. Response to Aza was not assessed until ≧3 weeks had passed since the last dose of myeloid growth factor.

Male and female MDS patients years of age with a diagnosis of RA, RARS, RAEB, RAEB-T, or CMML as defined by FAB classification criteria, and life expectancy ≧7 months were included. RA or RARS patients met at least 1 of the following criteria:

1) Hemoglobin <110 g/L with requirements for packed RBC transfusions;

2) Thrombocytopenia with platelet count <100×10⁹/L;

3) Neutropenia with absolute neutrophil count (ANC) <1.5×10⁹/L.

Patients had an ECOG Performance Status Grade of 0-3. Additionally, on laboratory screening, serum bilirubin level ≦1.5× the upper limit of normal (ULN) range; SGOT or SGPT level ≦2× ULN; and serum creatinine level ≦1.5× ULN were required. Only patients deemed unlikely to proceed to bone marrow transplantation or stem cell-transplantation following remission were enrolled.

Patients were excluded if they had secondary MDS, a history of AML, or other malignant disease. Also excluded were those with uncorrected red cell folate deficiency or vitamin B₁₂ deficiency.

All patients provided written, informed consent before study participation and the study protocol was approved by the appropriate institutional review boards (IRBs).

All patients who received Aza (intention to treat [ITT] cohort) were evaluable for safety. Patients were evaluated for efficacy by IWG 2000 criteria if they had completed ≧56 days of Aza treatment.

Efficacy was measured as rates of IWG-defined hematologic improvement (HI) as follows: Erythroid: Major: >2 g/dL increase if hemoglobin <11 g/dL at baseline, or transfusion independence for RBC transfusion-dependent patients; Minor: 1-2 g/dL increase if hemoglobin <11 g/dL at baseline, or 50% decreased transfusion requirement for RBC transfusion-dependent patients. Platelet: Major: ≧30,000/mm³ increase if platelets <100,000/mm³ pretreatment, or transfusion independence for platelet transfusion-dependent patients; Minor: ≧50% increase (>10,000/mm³ but <30,000/mm³) if <100,000/mm³ at baseline. Neutrophil: Major: ≧100% increase if <1500/mm³ pretreatment, or absolute increase >500/mm³ (whichever is greater); and Minor: ≧100% increase but <500/mm³ if <1500/mm³ pretreatment.

Additionally, rates of transfusion independence, defined as a transfusion-free period of ≧56 days in patients who were transfusion dependent or independent at baseline were assessed.

Number, proportion, and 95% confidence intervals (95% CI) for patients achieving HI were summarized for the evaluable patient population and for FAB-defined low-risk (RA and RARS) patients. HI rates were compared among the 3 alternative dosing schedules using Fisher's exact tests. Onset of HI by Aza treatment cycle was reported descriptively.

Number and percent (with 95% CI) of RBC and platelet transfusion-dependent patients at baseline who achieved transfusion independence were assessed in the group of all evaluable patients and in FAB-defined low-risk patients in each alternative dosing regimen. A comparison among the 3 dosing arms of patients transfusion-dependent at baseline who achieved transfusion independence for RBCs and/or platelets during treatment was performed using Fisher's exact test.

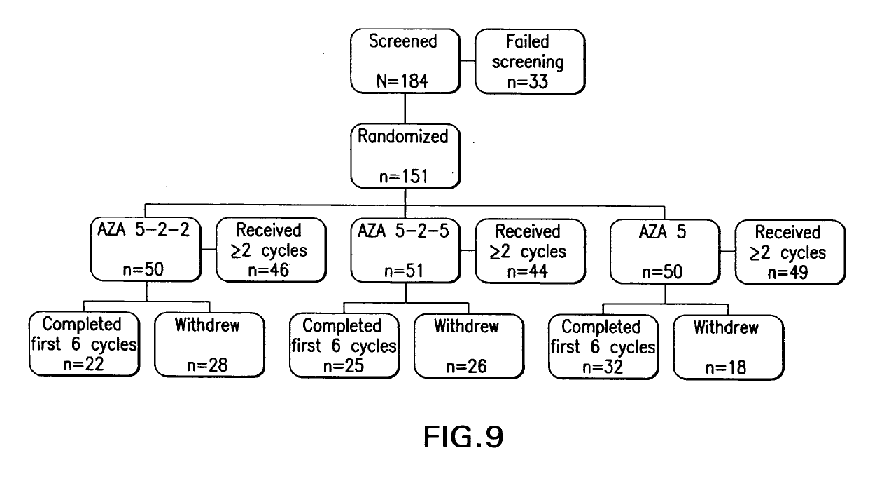

Of 184 patients screened, 151 (82%) were eligible and comprised the ITT cohort (FIG. 9). Patient demographic and disease characteristics at baseline are shown in Table 4. Most patients were RA/RARS (57%) or RAEB (30%) and ECOG status grade 0-1 (n=129, 85%). Of the ITT cohort, 3 patients did not receive study drug and were excluded from the safety-evaluable population (n=148). A total of 139 patients (92%) had ≧56 treatment days and comprised the efficacy-evaluable population.

TABLE 4
Patient Demographic and Disease Characteristics for All
Randomized Patients (N = 151) at Baseline

| | AZA 5-2-2 | AZA 5-2-5 | AZA 5 |
|---|---|---|---|
| Characteristic | N = 50 | N = 51 | N = 50 |
| Age, median (range) | 73 | 76 | 76 |
| | (37-88) | (54-91) | (47-93) |
| Gender, n (%) | | | |
| Male | 28 (56) | 37 (73) | 33 (66) |
| Female | 22 (44) | 14 (28) | 17 (34) |
| ECOG Status, n (%) | | | |
| Grade 0 | 19 (38) | 14 (28) | 12 (24) |
| Grade 1 | 23 (46) | 29 (57) | 32 (64) |
| Grade 2 | 5 (10) | 7 (14) | 3 (6) |
| Grade 3 | 3 (6) | 1 (2) | 3 (6) |
| RBC Transfusion Dependent | 24 (48) | 23 (46) | 25 (49) |
| Platelet Transfusion Dependent | 2 (4) | 0 | 3 (6) |
| FAB Classification, n (%) | | | |
| RA | 22 (44) | 21 (41) | 22 (44) |
| RARS | 7 (14) | 7 (14) | 7 (14) |
| RAEB | 14 (28) | 17 (33) | 14 (28) |
| RAEB-T | 1 (2) | 1 (2) | 2 (4) |
| CMMoL | 6 (12) | 5 (10) | 5 (10) |
| WHO Classification, n (%) | | | |
| RA | 19 (38) | 12 (24) | 15 (30) |
| RARS | 5 (10) | 6 (12) | 8 (16) |
| RCMD | 4 (8) | 13 (26) | 6 (12) |
| RCMD-RS | 2 (4) | 0 (0) | 0 (0) |
| RAEB-1 | 8 (16) | 10 (20) | 6 (12) |
| RAEB-2 | 9 (18) | 8 (16) | 10 (20) |
| MDS-Unknown | 1 (2) | 0 (0) | 1 (2) |
| Myeloproliferative | 0 (0) | 1 (2) | 0 (0) |
| Disorder | | | |
| Missing | 2 (4) | 1 (2) | 4 (8) |

CMMoL = chronic myelomonocytic leukemia;
FAB = French-American-British;
RA = refractory anemia;
RAEB = RA with excess blasts;
RAEB-T = RA with excess blasts in transformation;
RARS = RA with ringed sideroblasts;
RCMD = refractory cytopenia with multilineage dysplasia;
RCMD-RS = RCMD with ringed sideroblasts

Overall, 79 patients (52%) completed the 6 treatment cycles. Reasons for withdrawal (n=72) included adverse events (n=20), investigator opinion (n=16), withdrawal of consent (n=14), transformation to AML (n=7), disease progression (n=7), sponsor decision (n=3), death (n=3), and protocol deviation (n=2).

Hematologic Improvement

Numbers of patients with hematologic improvement (HI) (major and minor) are shown in Table 5. In the 3 alternative dosing groups, 5 (11%), 3 (7%), and 5 (10%) of patients experienced bilineage HI in the AZA 5-2-2, AZA 5-2-5, and AZA 5 groups, respectively, and 2 patients (4%) in each of the 3 alternative treatment groups experienced trilineage HI. Onset of HI occurred within the first 3 cycles for 87%, 88%, and 96% of patients in the AZA 5-2-2, AZA 5-2-5, and AZA 5 groups, respectively (Table 6).

TABLE 5
IWG (2000) Defined Hematologic Improvement (Evaluable Patients)

| | AZA 5-2-2 (N = 46) | AZA 5-2-5 (N = 44) | AZA 5 (N = 49) |
|---|---|---|---|
| Major HI | n (%) [95% CI] | n (%) [95% CI] | n (%) [95% CI] |
| Erythroid | | | |
| Major | 15 (33) [20, 48] | 17 (39) [24, 55] | 18 (37) [23, 52] |
| Minor | 1 (2) [0.1, 12] | 1 (2) [0.1, 12] | 1 (2) [0.1, 11] |
| Platelet | | | |
| Major | 10 (22) [11, 36] | 8 (18) [8, 33] | 9 (18) [9, 32] |
| Minor | 0 [0, 8] | 0 [0, 8] | 2 (4) [0.5, 14] |
| Neutrophil | | | |
| Major | 3 (7) [29, 100] | 4 (9) [40, 100] | 4 (8) [40, 100] |
| Minor | 0 [0, 8] | 0 [0, 8] | 2 (4) [0.5, 14] |
| Any HI* | 20 (44) [29, 59] | 23 (52) [37, 68] | 27 (55) [40, 69] |

Includes major and minor HI; patients counted only once for best response in an improvement category.

TABLE 6
Onset of IWG (2000) Defined Hematologic Improvement

| Onset | | | |
|---|---|---|---|
| of IWG- | AZA 5-2-2 (n = 22) | AZA 5-2-5 (n = 24) | AZA 5 (n = 28) |
| defined HI | n (%) | n (%) | N (%) |
| Cycle 1 | 9 (41) | 6 (25) | 15 (54) |
| Cycle 2 | 9 (41) | 8 (33) | 10 (36) |
| Cycle 3 | 1 (5) | 7 (29) | 2 (7) |
| Cycle 4 | 0 | 2 (8) | 1 (4) |
| Cycle 5 | 2 (9) | 1 (40 | 0 |
| Cycle 6 | 1 (5) | 0 | 0 |

Transfusion Independence

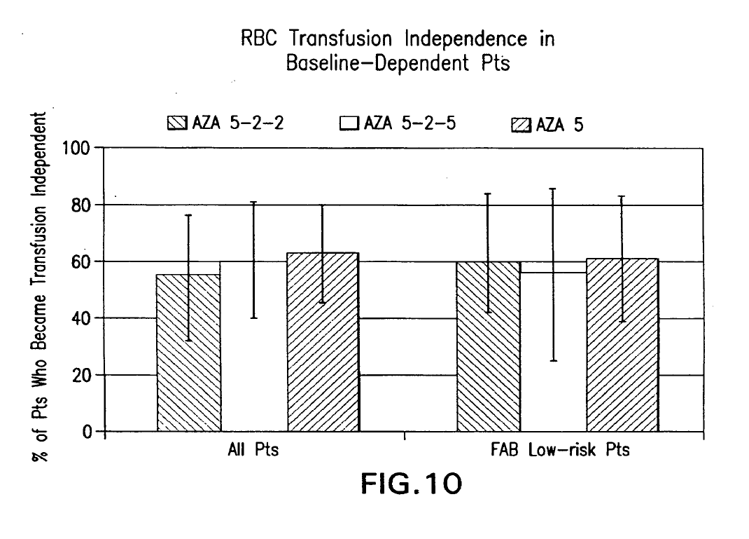

Proportions of all evaluable and FAB low-risk patients who were RBC transfusion-dependent at baseline and achieved transfusion independence during Aza treatment are shown in FIG. 10. Mean durations of RBC transfusion independence were 135 days, 138 days and 109 days in the AZA 5-2-2, AZA 5-2-5, and AZA 5 dosing arms, respectively. Proportions of RBC transfusion-dependent patients who achieved transfusion independence and retained independence at the end of cycle 6 (i.e., median transfusion independence duration not yet reached) were 100%, 92% and 63%, respectively. Of evaluable patients who were RBC transfusion independent at baseline, 67%, 79% and 68% of evaluable patients remained transfusion independent during the study in the AZA 5-2-2, AZA 5-2-5, and AZA 5 treatment arms, respectively, and 75%, 80%, and 64%, respectively, of FAB low-risk patients remained transfusion independent during the study.

Five patients were platelet transfusion-dependent at baseline (AZA 5-2-2 n=2, AZA 5 n=3), all of whom achieved transfusion independence during the study. Of patients who were platelet transfusion independent at baseline, ≧92% of each alternative dosing regimen group remained transfusion independent.

Safety and Tolerability

The three azacitidine alternative dosing regimens were generally well tolerated, with a majority of patients (52%) completing all 6 treatment cycles. Safety profiles were consistent among dosing arms, although the AZA 5 regimen appeared to be slightly better tolerated than the other 2 regimens. The most commonly reported hematologic AEs were neutropenia (38%), anemia (29%), thrombocytopenia (24%), and leukopenia (18%). The most commonly reported nonhematologic AEs were fatigue (93%), nausea (55%), injection site erythema (55%), injection site pain (54%), and constipation (51%). Grade 3 and 4 treatment-related AEs of special interest are listed in Table 7.

TABLE 7
Selected Grade 3/4 Adverse Events

| | AZA | | | |
|---|---|---|---|---|
| | 5-2-2 | AZA 5-2-5 | AZA 5 | Total |
| | (N = 50) | (N = 48) | (N = 50) | (N = 148) |
| Event | n (%) | n (%) | n (%) | n (%) |
| ≧1 Adverse Event | 42 (84) | 37 (77) | 29 (58) | 108 (73) |
| Hematologic Disorders | 33 (66) | 24 (50) | 17 (34) | 74 (50) |
| Anemia | 12 (24) | 7 (15) | 5 (10) | 24 (16) |
| Febrile neutropenia | 4 (8) | 4 (8) | 1 (2) | 9 (6) |
| Leukopenia | 7 (14) | 4 (8) | 4 (8) | 15 (10) |
| Neutropenia | 21 (42) | 15 (31) | 11 (22) | 47 (32) |
| Thrombocytopenia | 13 (26) | 7 (15) | 6 (12) | 26 (18) |
| Hemorrhagic Events | 4 (8) | 3 (6) | 1 (2) | 8 (5) |
| Hemorrhagic anemia | 0 | 0 | 1 | |
| GI hemorrhage | 1 (2) | 1 (2) | 0 | 2 (1) |
| Rectal hemorrhage | 0 | 1 (2) | 0 | 1 (1) |
| Epistaxis | 1 (2) | 0 | 0 | 1 (1) |
| Infections | 11 (22) | 14 (29) | 5 (10) | 30 (20) |
| Candida Sepsis | 0 | 0 | 1 (2) | 1 (1) |
| Cellulitis | 2 (4) | 1 (2) | 1 (2) | 4 (3) |
| Pneumonia | 0 | 4 (8) | 1 (2) | 5 (3) |
| Urinary tract infection | 0 | 3 (6) | 0 | 3 (2) |

Patients with at least 1 treatment-emergent AE that led to study discontinuation numbered 9 (18%), 10 (20%), and 7 (14%) in the AZA 5-2-2, AZA 5-2-5, and AZA 5 dosing arms, respectively. AEs that led to discontinuation included neoplasms (n=8), general disorders and administration site conditions (n=5), skin and subcutaneous tissue disorders (n=4), infections (n=3), GI disorders (n=3), blood and lymphatic system disorders (n=2), metabolism and nutrition disorder (n=2), injury (n=1), investigation (n=1).

Serious AEs were reported in 27 (54%), 19 (40%), and 15 (30%) of patients in the AZA 5-2-2, AZA 5-2-5, and AZA 5 dosing regimen groups, respectively. The majority of these reports involved blood or lymphatic system disorders (n=19, 13%), infections (n=31, 21%), and GI disorders (n=12, 8%).

The 3 alternative Aza dosing regimens had comparable efficacy, with response rates similar to those seen with the currently approved Aza dosing regimen. IWG-defined HI rates in this study ranged from 44% to 55% of evaluable patients, compared with IWG-defined HI rates of 23% to 36% in the 3 earlier CALGB studies. Similarly, 55% to 63% of all evaluable patients, and 56% to 61% of FAB-defined low-risk patients in this study who were RBC transfusion dependent at baseline achieved transfusion independence during the study, compared with 45% of patients in the pivotal CALGB study treated subcutaneously with the approved Aza dose regimen. The higher HI and transfusion independence rates in this study may reflect the participation of a higher proportion of low-risk MDS patients compared with the earlier Aza studies.

Based on its MOA, Aza becomes incorporated into RNA and DNA. Methylation in the gene-promotor region of DNA generally correlates with gene silencing. In cancer, hypermethylation is a mechanism for inactivation of tumor suppressor genes, including genes responsible for cell-cycle control, apoptosis, and DNA repair and differentiation. Incorporation of Aza into DNA results in dose- and time-dependent inhibition of DNA methyltransferase activity and such exposure results in the synthesis of hypomethylated DNA and re-expression of previously quiescent tumor suppressor genes.

In the present study, onset of HI was relatively rapid, occurring within the first 3 cycles for 87%-96% of patients across dosing arms. In this study, maintenance of treatment effect was evident by the continued duration of transfusion independence in patients who were RBC transfusion dependent at baseline: 63% to 100% of patients across dosing arms were transfusion independent at the end of cycle 6. A 12-month maintenance phase was added to this study, in this phase, continuing patients were randomized to AZA 5 (75 mg/m²/day SC×5 days) repeated every 28 days or to AZA 5 repeated every 42 days.

Longer Aza use (9 cycles at the currently recommended dosing regimen) was a prospective feature of study design for a recently reported multicenter, randomized, open-label survival trial of Aza in high-risk MDS patients. Results showed Aza plus BSC significantly prolongs survival compared with conventional care regimens (e.g., low-dose Ara-C or standard chemotherapy) plus BSC or BSC only. Although survival was not assessed in the present study, the WHO-classification based time-dependent prognostic scoring system (WPSS) identified transfusion requirements as predictive of survival and leukemic evolution in MDS patients at any stage in their disease course. Further study may elucidate whether survival benefits observed with consecutive 7-day dosing of Aza in high-risk MDS patients are also conferred to low-risk MDS patients receiving alternative dosing schedules of Aza.

The three azacitidine alternative dosing regimens were generally well tolerated with consistent safety profiles, which were similar to that observed with the approved Aza dosing regimen. The AZA 5 dosing regimen appeared to be somewhat better tolerated than the other alternative dosing regimens, which were more frequently administered and provided higher cumulative doses per cycle. Lower Aza doses are likely to be less myelosuppressive. More data are needed to draw conclusions regarding the relative benefit-risk ratios of the 3 alternative dosing regimens. For example, efficacy of the AZA 5 dosing regimen was comparable to the other 2 regimens, however, duration of RBC transfusion independence in baseline-dependent patients was somewhat shorter than in the other 2 dosing arms. With the fewest administration days, AZA 5 may offer the most convenient dosing schedule.

The benefits of alternative dosing schedules observed in the present study suggested clinicians may have flexibility in designing convenient and tolerable Aza treatment regimens for their individual patients.

### 6.3 Example 3

The study assessed usage patterns and transfusion requirements in patients enrolled in AVIDA, a longitudinal registry of patients with hematologic disorders receiving azacitidine, to further the understanding of current azacitidine treatment patterns in the community, identify common care procedures and concomitant treatments, and document transfusion requirements.

MDS are a heterogenous group of myeloid neoplasms characterized by ineffective hematopoiesis and peripheral cytopenias. Treatment decisions are often based on age, performance status (PS), cytopenias, IPSS classification, and MDS subtype. Patient-reported results from a few clinical trials suggest that MDS can have a negative effect on patient's quality of life (QoL) with responses to treatment having a positive effect.

In a Phase III study, patients treated with azactidine experienced significantly greater improvement in QoL compared with supportive care. Evaluation of QoL in MDS patients treated in community-based hematology clinics was not well characterized. Azacitidine was approved for a dosing schedule of 75 mg/m²/day subcutaneously (SC) for 7 days every 28 days. However, the dose and schedule of azactidine used in clinical practice varied. AVIDA was a unique, longitudinal, multicenter patient registry designed to prospectively collect data from community-based hematology clinics on the natural history and management of patients with MDS and other hematologic disorders, including acute myeloid leukemia, who are treated with azactidine.

Baseline demographics and disease characteristics were obtained at enrollment. Azacitidine treatment patterns, including dose and administration, transfusion requirements, and onset of transfusion independence (no transfusions for 56 days and have received 2 or more cycles of azacitidine) were recorded.

136 patients (95 males, 41 females; mean age, 73.7 yrs) with predominantly low-risk MDS were enrolled in AVIDA; 9 patients had AML. Median time from first MDS diagnosis was 2.8 months (mean, 13.8 months). Majority (82%) of patients had primary MDS and 77% had a baseline performance status of 0 or 1. Eighty (59%) patients had a history of RBC transfusion and 25 (18%) patients had a history of platelet transfusions; 47 (35%) patients had no history of any transfusion. Treatment data were available for 126 patients. A total of 360 cycles (median, 2; range, 1-14) of azacitidine were administered (46% via subcutaneous injection). The most common dose and schedule was 75 mg/m² (81%) at 5 days on treatment (53%). Seventy patients with available prior transfusion requirement data received at least 2 cycles of azacitidine; 81% (22/27) of patients without a history of transfusion achieved transfusion independence after a median of 4 cycles and 37% (16/43) of patients with a history of transfusions achieved transfusion-independence after a median of 6 cycles.

Based on data from the first 136 patients from AVIDA, the characterization of azacitidine treatment patterns in the community-based setting began to emerge. Early AVIDA data suggested that alternative dosing regimens were efficacious, in accordance with azacitidine clinical trials, and azacitidine allowed patients to maintain or achieve transfusion-independence.

### 6.4 Example 4

The study examined treatment of high-risk MDS patients with −7/del(7q) with azacitidine (AZA) vs. with conventional care regimens (CCR) and assessed the effects on overall survival. −7/del(7q) is associated with poor prognosis in MDS. This analysis assessed the effect of AZA on OS in this subgroup of high-risk MDS patients with −7/del(7q).

Primary inclusion criterion for the Phase III study was high-risk MDS (FAB RAEB, RAEB-T, or CMML and IPSS Int-2 or high). Patients were randomized to AZA (75 mg/m²/d×7d, q28d) or CCR. CCR comprised 3 treatments: BSC only (transfusions, antibiotics, G-CSF for neutropenic infection); low-dose ara-C (20 mg/m²/d×14d, q28d); or induction chemotherapy (7+3 regimen). No erythropoietin was allowed.

At baseline (BL), 57 (30 AZA, 27 CCR) of 358 patients in the total population had −7/del(7q), 35% had −7/del(7q) alone and 65% had −7/del(7q) as part of complex karyotype. BL characteristics were balanced in the 2 arms: 70% male, median age, 69 years. The median Kaplan Meier difference in OS for AZA vs. CCR was 8.4 months, a significant improvement (3-fold) over CCR (see Table 8). The hazard ratio (HR) was 0.33 (95% CI: 0.16-0.68) indicating a 67% reduced risk of death in the AZA arm, comparing with an HR of 0.58 for the OS improvement with AZA vs. CCR with all cytogenetic subtypes in the phase III trial. At 2 years, a 4-fold OS advantage was observed in the AZA arm with 33% of patients alive vs. 8% in the CCR arm (p=0.03). Secondary endpoints support the OS advantage (see Table 8). Significantly higher IWG 2000 response rates (CR+PR) were seen in patients with −7/del(7q) alone (64% vs. 11%, p=0.03) or complex (21% vs. 0; p=0.02) with AZA vs. CCR, and compared favorably to the overall AZA group with IPSS good and intermediate cytogenetics. AZA was generally well tolerated.

Patients with complete or partial chromosome 7 deletions, who have a particularly poor outcome with traditional management strategies, experienced the greatest overall survival improvement with azacitidine corresponding to a 67% reduction in risk of death (hazard ratio=0.33). The phase III subgroup analysis indicated the disease modifying effect of AZA extending to unfavorable cytogenetic patterns including −7/del(7q), and suggested AZA may represent the treatment of choice for this otherwise poor prognosis subset.

TABLE 8
OS, CR, PR, HI and transfusion independence (TI) in RBC-
dependent −7/del(7q) patients at BL (n/N, %)

| | OS | | | | | |
|---|---|---|---|---|---|---|
| | (mos) | CR + PR | CR | RBC TI | HI-E | HI-P |
| AZA | 13.1 | 13/30, 43 | 8/30, 27 | 12/21, 57 | 13/26, 50 | 10/20, 50 |
| CCR | 4.6 | 1/27, 4 | 1/27, 4 | 0/19, 0 | 0/24, 0 | 2/25, 8 |
| P | 0.003 | 0.0005 | 0.03 | <0.0001 | <0.0001 | 0.002 |
| value | | | | | | |

### 6.5 Example 5

Azacidine (AZA) extended overall survival in higher risk MDS without necessity for complete remission.

The importance of complete remission (CR) to extend survival was unclear—clinical validation in MDS was lacking. This analysis evaluated effects of AZA vs. conventional care regimens (CCR) on 1-year survival according to IWG 2000 defined response categories in the AZA-001 study.

MDS patients with FAB RAEB, RAEB-T, or CMML, and IPSS Int-2 or High Risk, were included. Patients were randomized to AZA (75 mg/m²/d SC×7d q 28d; n=179)+best support care (BSC; transfusions, antibiotics, and G-CSF for neutropenic infection) or to CCR+BSC (n=179). CCR+BSC included: low-dose ara-C (20 mg/m2/d×14d q 29d), standard chemotherapy (7+3 regimen), or BSC only. Erythropoietin was not allowed. One-year survival rates were determined for all treated patients in each arm, and for AZA subsets according to IWG-defined CR or partial remission (PR), stable disease (SD) or hematologic improvement (HI) as best response, or disease progression (DP).

AZA maintained a significant survival benefit vs. CCR with exclusion of CR patients from the analysis (hazard ratio OS=0.65, 95% CI: 0.48, 0.88). The one year survival rates were significant higher in AZA-treated patients than in CCR-treated patients: 68.2% vs. 55.6%, respectively (p=0.015). When analyzed by IWG 2000 best response, all response categories including SD showed an OS benefit with AZA treatment: CR (96.7%), PR (85.5%), HI (96.0%), or SD (73.3%), while only 28.6% of AZA patients with DP were alive at one year.

AZA as a disease-modifying agent improved one year OS regardless of IWG 2000 best response. The data from this study was the first to show that achievement of CR was not an obligate state for extended survival in higher risk MDS.

### 6.6 Example 6

The study assessed the effect of azacitidine (aza) versus low-dose ara-c (ldac) on overall survival (OS), hematologic response, transfusion independence, and safety in patients with higher risk MDS, to assess OS, response, transfusion independence and safety in a subgroup analysis comparing patients receiving AZA vs. LDAC.

Higher risk MDS patients (FAB: RAEB, RAEB-T, CMML; IPSS: Int-2, High) were enrolled. Before randomization, investigators selected the most appropriate of 3 CCR (best supportive care only, LDAC [20 mg/m²/d×14d every 28 days for ≧4 cycles], or intensive chemotherapy) for all enrolled patients. Then, if randomized to AZA, patients received AZA (75 mg/m²/d SC×7d every 28 days for ≧6 cycles) regardless of investigator selection; if randomized to CCR, patients received their investigator-selected treatment. All regimens were continued until study end, relapse, progression, unacceptable toxicity, or AML transformation. For this subgroup analysis, OS, hematologic response (IWG 2000), transfusion independence (≧56 days) were compared between the AZA and LDAC groups. This subgroup analysis was conducted in the 94 patients selected by investigators to receive LDAC treatment. Per randomization, 45 were treated with AZA and 49 with LDAC. These patients groups were well matched because both were selected for LDAC therapy. This was an intent-to-treat subgroup analysis using Cox proportional hazard modeling stratified by IPSS and FAB subtypes, adjusting for baseline ECOG, RBC transfusions, FAB subtype, presence of −7/del(7q), LDH, and hemoglobin. Median OS was analyzed using Kaplan-Meier methods. Erythropoiesis-stimulating agents were disallowed. All patients gave informed consent.

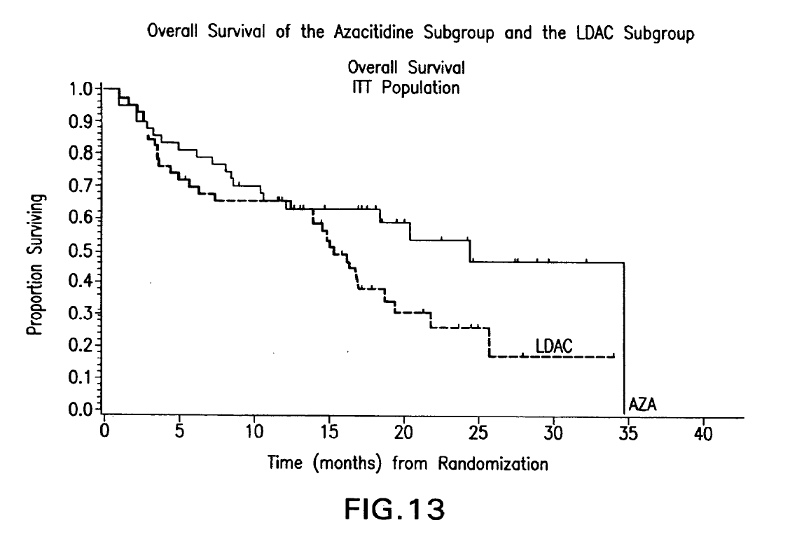

Baseline characteristics were similar between the 2 treatment groups. AZA was administered for a median of 9.0 cycles (range: 1-39), LDAC for 4.5 cycles (range: 1-15). Higher rates of early discontinuation were observed in the LDAC group (67%) due to withdrawal of consent, adverse events, and progression compared with the AZA group (39%). Median OS was 24.4 months (95% CI: 12.0-34.7) versus 15.3 months (95% CI: 13.9-18.8) in the AZA and LDAC groups, respectively (FIG. 13), for a difference of 9.2 months, hazard ratio: 0.38 (95% CI: 0.21-068, p=0.001). CR+PR rates were 31.1% versus 12.2% in the AZA and LDAC groups (p=0.042), respectively, and HI (major +minor) was observed in 53.3% and 25.0% (p=0.006). Transfusion independence in baseline-dependent patients was observed in 45% and 13% of patients in the AZA and LDAC groups, respectively (p=0.011). Higher rates of grade 3-4 thrombocytopenia and anemia were seen in the LDAC group versus the AZA group. Deaths during study were higher in the LDAC group versus the AZA group: 59% versus 45%, respectively.

Azacitidine significantly prolonged OS with significant improvement in clinical response and transfusion independence compared with LDAC and was better tolerated. Azacitidine should be considered first-line therapy compared with LDAC in higher risk patients with MDS.

### 6.7 Example 7

Azacitidine (aza) prolonged overall survival (OS) vs. conventional care regimens (CCR) in western Europe in higher risk MDS despite inter-country treatment selection differences.

This pooled, subgroup analysis assessed treatment pre-selections across five countries in the western EU, which enrolled 70% of the total AZA-001 patient population, to see if these pre-randomization selections affected the consistency of the overall OS findings across the countries (i.e., France, Germany, Italy, Spain, UK, Sweden, Greece, Netherlands).

For higher risk MDS patients (FAB-defined as RAEB, RAEB-T, or CMML; IPSS of Int-2 or High), prior to randomization, investigators preselected the most appropriate treatment for all patients from 3 conventional care regimens (CCR: best supportive care [BSC], low-dose ara-C [LDAC, 20 mg/m²/d×14d every 28 days for ≧4 cycles], or intensive chemotherapy [IC, 7+3 regimen]). Patients were then randomized to AZA or CCR. Those randomized to AZA received it at 75 mg/m²/d×7d, every 28 days for ≧6 cycles regardless of investigator selection; patients randomized to CCR received their investigator-selected treatment. Investigator selection and OS survival were compared by practice patterns across the five highest enrolling countries in Western EU: France, Germany, Italy, Spain, and the UK.

Overall, 252 patients were enrolled across the five EU countries. Investigator selection showed profound selection differences across the countries. Pooled results from France and the UK showed the highest preselection for LDAC (74% [62/84]) with 26 patients ultimately receiving AZA per randomization to AZA and 36 receiving LDAC per randomization to CCR. Pooled results from the UK, Italy, and Spain showed the highest preselection for BSC prior to randomization (82% [137/168]) with 68 patients receiving AZA per randomization to AZA and 69 receiving BSC per randomization to CCR. Survival analysis pooling France with the UK (where LDAC selection was highest) showed an OS advantage for the AZA group versus the CCR group similar to that observed in the overall AZA-001 OS analysis. Survival analysis pooling results from Germany, Italy, and Spain (where BSC selection was highest) also showed an OS advantage for the AZA group versus CCR that was highly similar to that observed in the overall OS findings (Table 9). Comparison of OS results in the pooled LDAC group (France/UK, n=36) with the pooled BSC group (Germany, Italy, Spain, n=69) showed no differences: 16.9 months versus 16.6 months (HR: 0.95; 95% CI: 0.55-1.62; log-rank p=0.843).

Independent of investigator treatment selection preferences for LDAC in France and the UK and for BSC in Germany, Italy, and Spain, differences in median OS between the AZA and CCR groups remained statistically significant and consistent with that demonstrated in the overall trial. Treatment with LDAC or BSC provided comparable outcomes with no survival benefit.

TABLE 9
OS (Median Months) in France/UK (LDAC Driven) and
Germany/Italy/Spain (BSC Driven) Compared with the
Overall AZA-001 Findings

| | OS | | | | Log- |
|---|---|---|---|---|---|
| Country | AZA | OS CCR | Difference | HR (95% CI) | rank P |
| France + | 24.5 | 16.4 | 8.0 | 0.44 (0.23-0.85) | 0.014 |
| UK | | | | | |
| Germany + | 25.1 | 16.6 | 8.6 | 0.63 (0.02-0.57) | 0.031 |
| Italy + Spain | | | | | |
| Overall | 24.4 | 15.0 | 9.4 | 0.58 (0.43-0.77) | 0.0001 |
| AZA-001 | | | | | |

### 6.8 Example 8

Effective treatment of elderly patients with acute myelogenous leukemia (AML) remains a challenging task. Elderly patients with AML usually respond poorly to standard induction chemotherapy. Response rates in elderly patients are in the range of 30-50% compared to 80-90% in younger patients. Moreover, prolonged hospitalization with treatment related mortality as high as 30% is typical in this older population. In a prior retrospective analysis done at our institution, azacitidine showed an overall response rate of 60% with limited toxicity when administered to patients older than 55 years of age with AML.

This is a prospective, phase II open label study using azacitidine in patients ≧60 years with AML. Inclusion criteria: Newly diagnosed AML (de novo or secondary, WHO criteria), and ECOG≦2. Promyelocytic (M3) phenotype was excluded. Patients with circulating blast count≧30,000/mcl were treated with hydroxyurea until<30,000/mcl. Azacitidine was given at a dose of 100 mg/m² subcutaneously for 5 consecutive days every 28 days until disease progression or significant toxicity. G-CSF was given to patients with neutropenia (ANC<1000/mcl) during all cycles excluding cycle one.

Eight patients had been enrolled to date. The mean age of patients was 74 (range: 64-82 years). The mean baseline ECOG performance score was 1 with a mean during treatment of 1. Mean baseline bone marrow blast count was 53% (range: 21-92%). Overall response rate was 75% (6/8): complete response (CR; n=2; 25%) and partial response (PR; n=4; 50%). The mean number of days on treatment was 117 (range: 4-247 days). The mean number of days hospitalized during therapy was 18 (range: 7-51 days) with the majority of therapy being given in the outpatient setting. The mean overall survival time from diagnosis for all patients was 180 days (range: 23-403). The mean overall survival time for responders was 200 days (range: 36-403). Three patients continue on therapy at 146 (PR), 153 (CR) and 247 (PR) days. Of the other responders, one went on to receive an allogeneic PBSCT, one died at 36 days from complications of a strangulated hernia, and one removed himself from study at 82 days (unconfirmed CR) to receive treatment closer to home. All patients were red blood cell (RBC) transfusion dependent at the start of the therapy. To date, two of the six responders (33%) became independent of RBC transfusion. Four patients were transfusion dependent for platelets at the start of therapy with two being non-responders and two achieving a PR. Non-hematological toxicity was limited to mild injection site skin reaction and fatigue in 63% (5/8) each. No treatment related deaths were observed.

Administration of subcutaneous azacitidine to elderly patients with acute myelogenous leukemia is a feasible and well-tolerated alternative to standard induction chemotherapy.

### 6.9 Example 9

Management of AEs is important to prevent early discontinuation of AZA, before therapeutic benefit may be achieved. This analysis evaluated the frequency of the most commonly reported (≧20% of patients) AEs with AZA by cycle, and the supportive care measures used to ameliorate AEs.

Patients with higher risk MDS (FAB-defined RAEB, RAEB-T, or CMML and IPSS Int-2 or High) were enrolled in the Phase III AZA-001 study described herein. Patients were randomized to AZA 75 mg/m²/d SC×7d q 28 days or to a conventional care regimen. AZA dosing cycles could be delayed based on hematologic recovery and AEs. Prophylactic G-CSF and erythropoietin were not allowed.

Of 179 patients randomized to AZA, 175 received the drug and were evaluable for safety (see Table 10). Median cycle length was 34 days (range: 15-92); 50% of AZA cycles were administered with no delays (at 28 days), 27% at 35 days, and 23% at >35 days. The majority of the most common AEs (≧20%), which included non-hematologic administration-related (injection site reactions, gastrointestinal) and hematologic events, were transient (median duration 13 days), non-serious, and resolved during the study. Less than 1% of AEs resulted in discontinuation of AZA and instead were commonly managed with delays in the next AZA cycle, concomitant medications, transfusions, and other measures. The median duration of injection site reactions was 12 days; none resulted in adjustment in AZA and <15% required treatment with concomitant medications (typically corticosteroids and/or antihistamines). The majority (95%) of gastrointestinal events were transient with a median duration of 1-4 days (diarrhea, nausea, vomiting) or approximately 1 week (constipation). No gastrointestinal events resulted in discontinuation of AZA and were more commonly managed (72%) with concomitant medications (e.g., anti-emetics, laxatives). Most hematologic AEs were transient (>86%), occurred during the first 1-2 cycles (median duration ˜2 weeks), and were mainly grade 3 or 4; however, ≦10% of patients experienced neutropenia, anemia, or thrombocytopenia that required hospitalization. The majority of hematologic events were managed with delays in the next AZA cycle (99%) or transfusions for anemia (87%) or thrombocytopenia (29%); <5% of patients discontinued due to a hematologic event. The median duration of fatigue and pyrexia was approximately 1 week; none of the events resulted in discontinuation or dose decrease of AZA and instead were managed by delay in the next AZA cycle in approximately 5% of patients. There were no cumulative or delayed toxicities.

The majority of the most common AEs (≧20%) in the AZA 001 study were transient (median duration 13 days), nonserious, and were managed by either dose delays for hematological events or supportive care measures. Clinicians should be alert to the onset, duration, and management of these events to allow patients to achieve maximum therapeutic benefit.

TABLE 10
Most Frequent (≧20% of Patients) Treatment-Emergent Adverse Events With
Azacitidine in AZA-001 Study

| | Percent of Patients Per Cycle | | | | |
|---|---|---|---|---|---|
| | Cycles | Cycles | Cycles | Cycles | Cycles |
| System Organ Class | 1-2 | 3-4 | 5-6 | 7-8 | 9-10 |
| Preferred Term* | (N = 175) | (N = 147) | (N = 130) | (N = 107) | (N = 89) |
| Patients with at least 1 | 94 | 79 | 65 | 65 | 65 |
| individual AE occurring | | | | | |
| in ≧20% of patients in | | | | | |
| the AZA group | | | | | |
| Blood and lymphatic | 75 | 54 | 42 | 36 | 36 |
| system disorders | | | | | |
| Anemia | 33 | 18 | 14 | 11 | 14 |
| Neutropenia | 50 | 31 | 28 | 19 | 20 |
| Thrombocytopenia | 54 | 30 | 25 | 20 | 21 |
| Gastrointestinal disorders | 62 | 42 | 25 | 27 | 30 |
| Constipation | 35 | 20 | 13 | 9 | 17 |
| Diarrhea | 12 | 8 | 4 | 5 | 5 |
| Nausea | 36 | 19 | 12 | 14 | 11 |
| Vomiting | 18 | 11 | 5 | 8 | 6 |
| General disorders and | 62 | 44 | 32 | 32 | 28 |
| administration site conditions | | | | | |
| Fatigue | 13 | 10 | 3 | 6 | 3 |
| Injection site erythema | 35 | 21 | 18 | 16 | 11 |
| Injection site reaction | 21 | 13 | 9 | 9 | 9 |
| Pyrexia | 16 | 6 | 4 | 6 | 7 |

*Multiple reports of the same preferred term for a patient are counted only once.

### 6.10 Example 10

MDS incidence increases with age resulting in limited treatment options particularly for those >75 years of age given the poor tolerability and ineffectiveness of cytotoxic therapies. This subgroup analysis of the Phase III AZA-001 study described herein compared the effects of AZA vs. CCR on OS, hematologic improvement (HI), transfusion independence (TI), and tolerability in patients ≧75 yrs of age.

Higher risk MDS (FAB: RAEB, RAEB-T, CMML and IPSS: Int-2 or High) patients were enrolled. All patients were pre-selected by site investigators—based on age, performance status, and co-morbidities—to receive 1 of 3 CCR: best supportive care only (BSC); low-dose ara-C (LDAC), or intensive chemotherapy (IC). Patients were then randomized to AZA (75 mg/m²/d SC×7d q 28d), or to CCR. Those randomized to AZA received AZA; those randomized to CCR received their pre-selected treatment. Randomization was stratified based on FAB subtype (RAEB and RAEB-T) and IPSS (Int-2 or High). Erythropoiesis stimulating agents were disallowed. OS was assessed using Kaplan-Meier (KM) methods and HI and TI per IWG 2000. To adjust for baseline imbalances, a Cox proportional hazards model was used, with ECOG status, LDH, number of RBC transfusions, Hgb, and presence or absence of −7/del(7q) at baseline as variables in the final model. Adverse events (AEs) were evaluated using NCI-CTC v. 2.0.

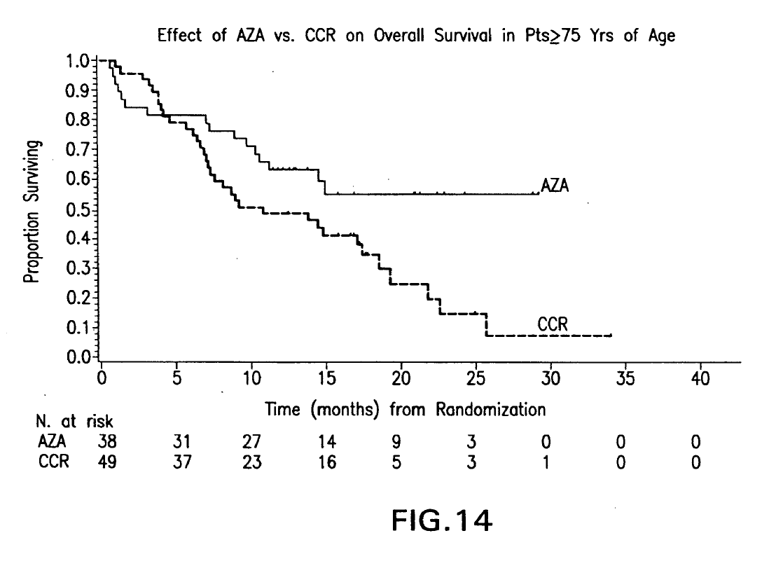

The majority of patients in this subgroup analysis randomized to CCR received BSC only, suggesting clinicians are reticent to use active treatment in this population. Of all enrolled patients (N=358, median age 69 yrs), 87 patients (24%) were ≧75 yrs of age (AZA n=38, CCR n=49 [BSC, n=33; LDAC, n=14; IC, n=2]). Similar to the overall AZA-001 results, treatment with AZA was associated with prolonged survival with KM median OS in the AZA group not reached at 17.7 months of follow-up, vs. KM median OS for CCR at 10.8 months (HR: 0.48 [95% CI: 0.26, 0.89]; p=0.0193). OS rates at 2 years were significantly higher in the AZA group vs. CCR: 55% vs 15% (p=0.0003) (FIG. 14). Two-fold more RBC transfusion-dependent patients at baseline in the AZA group achieved TI vs. CCR: 10/23 (44%) vs. 7/32 (22%), p=0.1386, respectively. Similarly, more patients in the AZA group achieved HI (major+minor) vs. CCR: 58% vs 39%, (p=0.0875), respectively. As previously reported, AZA was generally well tolerated. Anemia, neutropenia, and thrombocytopenia were seen in 42%, 66%, and 71% of patients in the AZA group, respectively, vs. 45%, 24%, and 39% in the CCR group, who were predominately receiving BSC only. Infections were reported by 79% and 60% of AZA and CCR patients, respectively. Discontinuations due to an AE occurred in 13% of AZA and 8% of CCR patients ≧75 yrs of age.

Data from this subgroup analysis indicate patients ≧75 yrs of age with higher risk MDS experience significantly prolonged 2-year OS and reduced risk of death receiving active treatment with AZA that is generally well tolerated.

### 6.11 Example 11

This analysis was conducted to assess the median number of AZA treatment cycles associated with achievement of first response, as measured by IWG 2000-defined CR, PR, or HI (major+minor). The number of treatment cycles to best response was also measured.

Patients (N=358) with higher risk MDS (FAB: RAEB, RAEB-T, or CMML, and IPSS: Int-2 or High) were included. Patients were randomized to AZA (75 mg/m²/d SC×7d q 28d) or to a conventional care regimen (CCR, n=179). AZA treatment was continued until disease progression (or unacceptable toxicity), regardless of hematologic response. Erythropoiesis stimulating agents were not allowed.

Of 179 AZA-treated patients, 91 (51%) achieved a CR, PR, or HI. For the 91 patients who achieved an IWG response, the median number of cycles to first response was 3 (range: 1-22), 81% of patients had achieved a first response at 6 cycles, and 90% had achieved a first response at 9 cycles. For 58% of responders (n=53), their first response was their best response; the remaining 42% (n=38) improved their response status over the next 1-11 treatment cycles, at a median of approximately 4 additional treatment cycles after their first response.

While many patients achieving a hematologic response with AZA do so in early treatment cycles, continued AZA dosing can further improve pt responses. In the AZA-001 study, the significant OS benefit was observed at a median of 9 treatment cycles (range 1-39). Continued AZA treatment is appropriate; in this study, patients continued to achieve a first response after being treated for more than 20 cycles, and more than 40% of those with a first response later achieved an improved response.

### 6.12 Example 12

Approximately one third of the patients enrolled in the phase III AZA-001 trial were RAEB-T (≧20%-30% blasts) (FAB) and now meet the WHO criteria for AML (See e.g., Blood 1999; 17:3835-49). Considering the poor prognosis (median survival <1 year) and the poor response to chemotherapy in these patients, this subgroup analysis evaluated the effects of AZA vs. CCR on OS and on response rates in patients with WHO AML.

The AZA-001 trial enrolled higher risk MDS patients (FAB: RAEB, RAEB-T, CMML and IPSS: Int-2 or High). Prior to randomization, site investigators pre-selected (based on age, performance status, and comorbidities) 1 of 3 CCR: best supportive care only (BSC); low-dose ara-C (LDAC), or intensive chemotherapy (IC). Patients were then subsequently randomized to AZA (75 mg/m²/d SC×7d q 28d) or CCR. OS was assessed by Kaplan-Meier (KM) methods and IWG AML criteria (See e.g., J Clin Oncol 2003; 21:4642-9) were used to assess morphologic complete remissions.

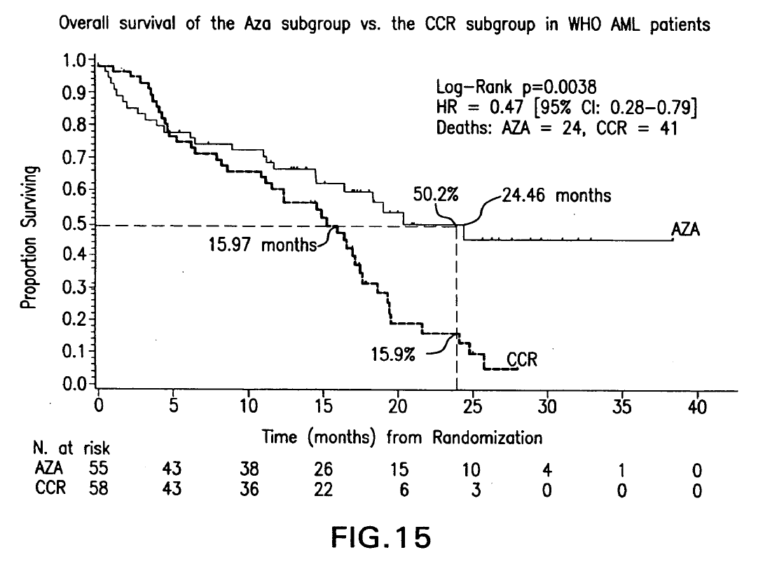

Of 358 enrolled patients, 113 met the definition for WHO AML (median: 23% blasts): 55 patients were randomized to AZA and 58 to CCR. AZA and CCR groups had comparable baseline demographic and clinical characteristics. Median age was 70 years, 46% had normal karyotype, 60% had 3 cytopenias, and 81% had IPSS High classification. Median follow-up for OS was 20.1 months. Median (min-max) number of treatment cycles was 8 (1-39) for AZA, 6 (2-19) for BSC, 5.5 (1-14) for LDAC, and 2.5 (1-3) for IC. KM median OS was 24.5 vs. 16.0 months, respectively, in the AZA and CCR groups, hazard ratio (HR)=0.47, 95% CI, 0.28 to 0.79, p=0.004, FIG. 15. OS rates at 2 yrs were 50% and 16%, respectively, in the AZA and CCR groups, p=0.0007. There was no statistical difference in the morphologic complete remission rate between groups (p=0.80). OS results in cytogenetic intermediate patients showed a significant HR favoring the AZA group (N=38) over CCR (N=43, HR: 0.47 [95% CI: 0.24, 0.91], p=0.024) but not in patients with unfavorable cytogenetics: AZA (N=14) vs. CCR (N=13, HR=0.66 [95% CI: 0.26, 1.68], p=0.381. WHO AML pt outcome measures showed significant benefits with AZA: fewer infections requiring IV antibiotics per pt-year in the AZA group (0.58) vs. CCR (1.14, RR=0.51, 95% CI 0.29, 0.78, p=0.003); and reduced rates of hospitalization in the AZA group (3.4 per pt-year) vs. CCR (4.3 per pt-year, RR=0.79, 95% CI 0.62, 1.00, p=0.028). Safety was consistent with previous reports.

AZA significantly prolongs OS with significant improvements in important pt outcomes in WHO AML patients.

### 6.13 Example 13

This analysis evaluated the predictive value of IWG responses of CR, partial remission (PR), hematologic improvement (HI), and stable disease (SD) on OS (death from any cause) in patients with higher risk MDS receiving AZA or a conventional care regimen (CCR) in the phase III AZA-001 study.

Patients with higher risk MDS (FAB: RAEB, RAEB-T, or CMML, and IPSS: Int-2 or High) were included. Patients were randomized to AZA (75 mg/m²/d SC×7d q 28d) or to 1 of 3 CCR: best supportive care only, low-dose ara-C (20 mg/m²/d×14d q 28d), or intensive chemotherapy (7+3 regimen). Randomization was stratified based on FAB subtype and IPSS. Erythropoiesis stimulating agents were disallowed. IWG 2006 responses were assessed and adjudicated by an independent review committee (IRC) of 4 international MDS investigators. Stratified Cox proportional hazards regression models were used to estimate hazard ratios (HR) and associated 95% confidence intervals (CI). Cox proportional hazards regression with stepwise selection was used to assess the baseline variables of sex, age, time since original MDS diagnosis, ECOG performance status (PS), number of prior RBC transfusions, number of prior platelet transfusions, Hgb, platelets, ANC, LDH, bone marrow blast percentage, and presence or absence of cytogenetic −7/del(7q) abnormality. The final model included ECOG PS, LDH, Hgb, number of RBC transfusions, and presence or absence of the cytogenetic −7/del(7q) abnormality. Each response, CR, PR, HI, and SD, was separately assessed as a time-dependent covariate in the final model. The responses were entered as a step function beginning when the response started and stopping when the response ended. To investigate the lag effect of the response over time, analyses were repeated with response end dates extended by 6 months.

A total of 358 patients were included (AZA n=179, CCR n=179). IRC-determined IWG response rates in the AZA and CCR groups, respectively, were 16% vs. 3% for CR (p<0.001), 1% vs. 0% for PR (p=0.46), 36% vs. 11% for HI (p<0.001), and 74% vs. 75% had SD (p=1.00). Median duration (days) of responses was significantly longer for AZA vs. CCR: 156 vs. 87 for CR; 217 vs. N/A for PR; 241 vs. 169 for HI; and 257 vs. 174 for SD. All outcomes, CR, PR, HI, and SD, were highly predictive of OS (p<0.001 for all comparisons). For CR and PR, the hazard rate was 0 (i.e., no deaths occurred during a CR or PR). For HI, the HR was 0.03 (95% CI: 0.00, 0.20), and for SD, the HR was 0.15 (95% CI: 0.10, 0.23). For the 6-month lag analyses, the HR was 0.25 (95% CI: 0.08, 0.81), p=0.021 for CR; hazard rate was 0 for PR; HR was 0.28 (95% CI: 0.17, 0.48), p<0.001 for HI; and HR was 0.41 (95% CI: 0.30, 0.57), p<0.001 for SD.

There were significantly higher rates of CR and HI with significantly longer durations of CR, PR, HI and SD for AZA vs. CCR. Traditional responses of CR and PR were not obligate endpoints for prolonged OS in these higher risk MDS patients.

### 6.14 Example 14

Preparative regimen dose intensity has frequently failed to improve outcomes of relapsed/refractory AML/MDS. It is possible that maintenance therapy after HSCT may provide an “adjuvant” for the allogeneic graft-versus-leukemia effect, and decrease the likelihood of recurrence. To begin assessment of whether AZA maintenance will reduce relapse rates, this study involved a phase I clinical trial to determine the safest dose and schedule combination.

Eligible were patients with AML or high-risk MDS not in 1st complete remission (CR), not candidates for ablative regimens due to age or co-morbidities. Conditioning regimen was gemtuzumab ozogamicin 2 mg/m² (day −12), fludarabine 120mg/m², and melphalan 140 mg/m². GVHD prophylaxis was tacrolimus/mini-methotrexate. Recipients of unrelated donor HSCT received ATG. The study was performed with 4 AZA doses: 8, 16, 24 and 32 mg/m² daily×5 starting on day +42, and given for 1-4 28-day cycles (schedule). An outcome-adaptive method was used to determine both dose and schedule (number of cycles): patients were assigned to a dose/schedule combination chosen on the basis of the data (organ and hematologic toxicity) from all patients treated previously in the trial. Patients in CR on transplant day +30, with donor chimerism, without grade III/IV GVHD, platelet >10,000/mm³ and ANC >500/mm³ were eligible to receive AZA. The methylation status of long interspersed nuclear elements (LINE) was analyzed by pyrosequencing and used as a surrogate marker of global DNA methylation in mononuclear cells of 38 patients that received AZA.

Eighty patients were transplanted; 44 (56%) were eligible to receive AZA: 2 patients refused, and 42 patients (4 too early) were assigned a dose and schedule and received the drug. Eighty-eight cycles of AZA were delivered at 8 (n=7 patients), 16 (n=5) and 24 mg/m² (n=21 patients) and 32 mg/m² (n=9 patients). AZA-associated (or possibly associated) toxicities were grade I/II or III thrombocytopenia (n=5, n=2, in association with MMF and sirolimus), grade I nausea (n=5), grade II fatigue (n=2), grade III elevation of transaminases (n=1, when drug given in association with posaconazole and pentamidine), erythema of the conjunctiva (n=1), pruritus (n=1), grade I confusion (n=2), retina hemorrhage (possibly pre-existing, but led to drug withdrawal after 1 cycle), grade II creatinine elevation (n=1, in the context of multiple nephrotoxic drugs), oral ulcers (n=1), papilledema (n=1, unclear if associated with drug), and pulmonary hemorrhage (n=1; patient receiving a second HSCT was found to have fungal pneumonia and hepatic VOD during the first AZA cycle, and evolved with thrombocytopenia and bleeding). Reversible thrombocytopenia was documented more often with 32 mg/m². Infections were as follows: bacteremia, n=5; pneumonia or other respiratory infection, n=6; C. difficile-associated diarrhea, n=2; VRE colonization, n=2; polyoma-related hemorrhagic cystitis, n=1; influenza/parainfluenza, n=2.

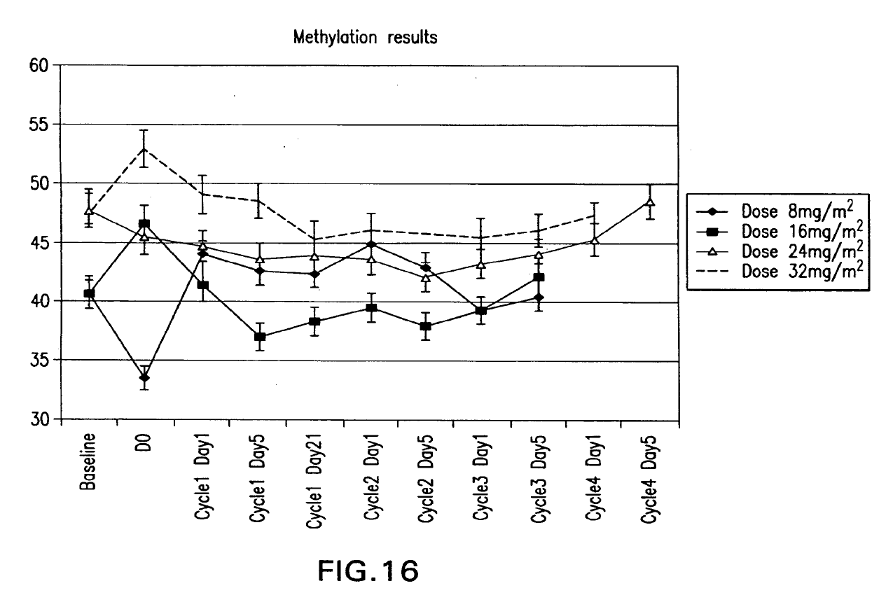

There was no indication of an effect on acute or chronic GVHD incidence. Patients relapsed, 2 while on AZA (16 and 24 mg/m²). Most patients were 100% donor chimeras at the start of AZA. Median follow-up of alive patients is 13 months (range, 3-31; n=26). 12 patients died, 8 of recurrence, 2 of GVHD, 1 of pneumonia and 1 of unknown causes. Day +100 non-relapse mortality was 6%. Four patients died within the first 100 days post HSCT: 2 of relapse, 1 of pneumonia e 1 of GVHD. Actuarial 1-year event-free and overall survival is 58% and 72%, respectively. Mean LINE methylation results are shown in FIG. 16. No dose was found to affect global methylation in a statistically significant way. The trial design reached the dose of 32 mg/m² and the maximum number of cycles (n=4), but thrombocytopenia prevented escalation to the next dose level (40 mg/m²).

AZA at 32 mg/m² is safe and can be administered for at least 4 cycles to a population of heavily pre-treated patients with co-morbidities. The safety profile indicates that longer periods of administration merit investigation. This study supports the initiation of a randomized, controlled study of AZA for one year versus best standard of care (i.e., no maintenance therapy) for similarly high-risk patients with AML or MDS.

### 6.15 Example 15

This study evaluates gene methylation biomarkers and prolonged survival in patients with certain MDS (e.g., higher risk MDS) treated with azacitidine. Hypomethylation is believed to be a molecular mechanism of action of azacitidine; accordingly, research on the effect of methylation status particular genes, and of gene combinations, is conducted. Both DNA methylation and RNA methylation are contemplated as potential biomarkers.

A study is performed to examine whether baseline DNA and/or RNA methylation levels influence overall survival (OS) as well as the interaction between gene promotor methylation levels and treatment (e.g., azacitidine or CCR). Methylation is determined for 5 genes previously evaluated in MDS or AML: CDKN2B (p15), SOCS1, CDH1 (E-cadherin), TP73, and CTNNA1 (alpha-catenin), in pre-treatment bone marrow aspirates of patients enrolled in a clinical study using quantitative real-time methylation specific PCR (qMSP). The influence of methylation on OS is assessed using Cox proportional hazards models and Kaplan-Meier (KM) methodology.

The number of patients (for azacitidine and CCR) having nucleic acid sufficient for analysis of these 5 genes is determined. Methylation is detected in a specific percentage of patients for CDKN2B, SOCS1, CDH1, TP73, and CTNNA1. Differences in methylation levels between the treatment arms are determined. The OS benefit for azacitidine treatment is determined for patients who are positive and negative for methylation at these 5 genes. It is determined whether the presence of methylation is associated with improvement in OS in the CCR group (prognostic indicator of good outcome). The existence and magnitude of any effect is compared to the azacitidine group, which may suggest an interaction between DNA and/or RNA methylation and treatment.

OS improvement is assessed with azacitidine treatment in patients with methylation at any of these 5 genes, and HR of death for methylation is determined. The frequency of methylation of particular genes allows for examination of the influence of methylation level on OS and treatment effect. For example, for particular genes, lower levels of methylation may be associated with the longest OS and the greatest OS benefit from azacitidine treatment, compared with the absence of methylation. Influence of methylation level on OS may be assessed in each IPSS cytogenetic subgroup (good, intermediate, and poor). For example, the influence of methylation on OS may be strongest in the “poor” risk group, where risk of death is greatest.

Such data and analysis may indicate, e.g., that patients with lower levels of methylation may derive greater benefit from azacitidine. Molecular biomarkers may be important in MDS, e.g., as indicators of disease prognosis and predictors of response to epigenetic therapy.

While the examples have been particularly shown and described with reference to a number of embodiments, it would be understood by those skilled in the art that changes in the form and details may be made to the various embodiments disclosed herein and that the various embodiments disclosed herein are not intended to act as limitations on the scope of the claims. All patents, publications, and other references cited herein are incorporated by reference herein in their entireties.

What is claimed is:

1. A method of treating a higher risk myelodysplastic syndrome, which comprises administering to a patient having a higher risk myelodysplastic syndrome a therapeutically effective amount of a cytidine analog.

2. The method of claim 1, wherein the higher risk myelodysplastic syndrome is Intermediate-2 or High risk in international prognostic scoring system (IPSS), or refractory anemia with excess blasts, refractory anemia with excess blasts in transformation, or chronic myelomonocytic leukemia having 10-29% marrow blasts.

3. The method of claim 1, wherein the cytidine analog is selected from the group consisting of 5-aza-2′-deoxycytidine, 5-azacytidine, 5-aza-2′-deoxy-2′,2′-difluorocytidine, 5-aza-2′-deoxy-2′-fluorocytidine, 2′-deoxy-2′,2′-difluorocytidine, cytosine 1-β-D-arabinofuranoside, 2(1H) pyrimidine riboside, 2′-cyclocytidine, arabinofuanosyl-5-azacytidine, dihydro-5-azacytidine, N⁴-octadecyl-cytarabine, and elaidic acid cytarabine.

4. The method of claim 1, wherein the cytidine analog is 5-azacytidine.

5. The method of claim 1, wherein the cytidine analog is administered subcutaneously.

6. The method of claim 5, wherein 5-azacytidine is administered in an amount of 75 mg/m²/day for seven days every 28 days.

7. The method of claim 1, wherein the cytidine analog is administered orally.

8. A method of selecting a patient diagnosed with a myelodysplastic syndrome for treatment with a cytidine analog, comprising assessing a patient diagnosed with a myelodysplastic syndrome for having higher risk, and selecting a patient for treatment with 5-azacytidine where the patient's myelodysplastic syndrome is assessed as having higher risk.

9. The method of claim 8, wherein a higher risk myelodysplastic syndrome is Intermediate-2 or High risk in international prognostic scoring system (IPSS), or refractory anemia with excess blasts, refractory anemia with excess blasts in transformation, or chronic myelomonocytic leukemia having 10-29% marrow blasts.

10. The method of claim 8, wherein the cytidine analog is selected from the group consisting of 5-aza-2′-deoxycytidine, 5-azacytidine, 5-aza-2′-deoxy-2′,2′-difluorocytidine, 5-aza-2-deoxy-2′-fluorocytidine, 2′-deoxy-2′,2′-difluorocytidine, cytosine 1-β-D-arabinofuranoside, 2(1H) pyrimidine riboside, 2′-cyclocytidine, arabinofuanosyl-5-azacytidine, dihydro-5-azacytidine, N⁴-octadecyl-cytarabine, and elaidic acid cytarabine.

11. The method of claim 8, wherein the cytidine analog is 5-azacytidine.

12. The method of claim 8, wherein the cytidine analog is administered subcutaneously.

13. The method of claim 12, wherein 5-azacytidine is administered in an amount of 75 mg/m²/day for seven days every 28 days.

14. The method of claim 8, wherein the cytidine analog is administered orally.

15. A method of improving survival of a patient having higher risk myelodysplastic syndrome, which comprises administering to a patient having a higher risk myelodysplastic syndrome a therapeutically effective amount of a cytidine analog.

16. The method of claim 15, wherein a higher risk myelodysplastic syndrome is Intermediate-2 or High risk in international prognostic scoring system (IPSS), or refractory anemia with excess blasts, refractory anemia with excess blasts in transformation, or chronic myelomonocytic leukemia having 10-29% marrow blasts.

17. The method of claim 15, wherein the cytidine analog is selected from the group consisting of 5-aza-2′-deoxycytidine, 5-azacytidine, 5-aza-2′-deoxy-2′,2′-difluorocytidine, 5-aza-2′-deoxy-2′-fluorocytidine, 2′-deoxy-2′,2′-difluorocytidine, cytosine 1-β-D-arabinofuranoside, 2(1H) pyrimidine riboside, 2′-cyclocytidine, arabinofuanosyl-5-azacytidine, dihydro-5-azacytidine, N4-octadecyl-cytarabine, and elaidic acid cytarabine.

18. The method of claim 15, wherein the cytidine analog is 5-azacytidine.

19. The method of claim 15, wherein the cytidine analog is administered subcutaneously.

20. The method of claim 19, wherein 5-azacytidine is administered in an amount of 75 mg/m²/day for seven days every 28 days.

21. The method of claim 15, wherein the cytidine analog is administered orally.

22. A method for identifying a patient diagnosed with a myelodysplastic syndrome having an increased probability of obtaining improved overall survival following azacitidine treatment.

23. The method of claim 22, which comprises analyzing methylation levels of the patient's nucleic acid.

24. The method of claim 23, wherein the nucleic acid is DNA.

25. The method of claim 23, wherein the nucleic acid is RNA.

26. The method of claim 23, wherein lower methylation levels indicate an increased probability of obtaining improved overall survival following azacitidine treatment.

27. The method of claim 23, which comprises analyzing the methylation level of a gene selected from CDKN2B (p15), SOCS1, CDH1 (E-cadherin), TP73, and CTNNA1 (alpha-catenin).

28. The method of claim 23, in which the patient's increased probability of obtaining improved overall survival following azacitidine treatment is used to plan or adjust the patient's azacitidine treatment.

29. The method of claim 22, in which the increased probability is a 10% greater probability.

30. The method of claim 22, in which the increased probability is a 50% greater probability.

31. The method of claim 22, in which the increased probability is a 100% greater probability.

32. The method of claim 22, in which the increased probability is a 200% greater probability.

33. A method for evaluating the influence of gene methylation on prolonged survival in patients diagnosed with a myelodysplastic syndrome.
